(19) United States
(12) Patent Application Publication
(10) Pub. No.: US 2021/0332095 A1
(43) Pub. Date: Oct. 28, 2021

(54) INFECTION-INDUCED ENDOTHELIAL AMYLOID COMPOSITIONS AS ANTIMICROBIALS

(71) Applicant: University of South Alabama, Mobile, AL (US)

(72) Inventors: Sarah Voth, Mobile, AL (US); Troy Stevens, Mobile, AL (US); Ron Balczon, Mobile, AL (US); Kyle Adam Morrow, Mobile, AL (US)

(21) Appl. No.: 17/273,940

(22) PCT Filed: Sep. 6, 2019
(86) PCT No.: PCT/US2019/049980

§ 371 Date: Mar. 5, 2021

Related U.S. Application Data
(60) Provisional application No. 62/727,840, filed on Sep. 6, 2018.

(51) Int. Cl. C07K 14/47; A61K 38/17; A61K 39/395; A61P 31/04; C12P 21/02; C07K 16/18

(52) U.S. Cl. CPC C07K 14/4711; A61K 38/1716; A61K 39/3955; C07K 2317/76; C12P 21/02; C07K 16/18; A61P 31/04

(57) ABSTRACT

The present disclosure relates to compositions and methods for the production of antimicrobial amyloid compositions, and further relates to use of such antimicrobial preparations for the treatment of subjects having drug-resistant microbial infections. Advantageous and/or therapeutic amyloid oligomer immunodepletion methods are also disclosed.

### STATEMENT REGARDING FEDERALLY SPONSORED RESEARCH

This invention was made with government support under Grant Nos. HL66299, HL60024 and HL076125, awarded by the National Institutes of Health. The government has certain rights in the invention.

### FIELD OF THE INVENTION

The invention relates generally to compositions, methods and kits for the treatment of diseases or disorder, using amyloids present in supernatants of certain infected cells.

### BACKGROUND OF THE INVENTION

The mortality rate in critically ill patients has improved significantly over the past 20 years, mostly due to advanced ventilation strategies and hemodynamic support. However, patients who survive critical illness have staggering rates of morbidity and mortality in the aftermath of their intensive care unit stay. This problem is notably worse in patients who have developed nosocomial infections, such as pneumonia. Nosocomial pneumonia is associated with an excess hospital mortality of more than 10%. The 30-day hospital readmission rate in that patient population is over 20%. One-year survival in intensive care unit patients who developed nosocomial pneumonia is only ˜50%, which further decreases to 30% five years after intensive care unit admission. Healthcare utilization is also significantly increased for survivors. Indeed, in patients who survive beyond this first post-intensive care unit year, cardiovascular, endocrine and neurocognitive dysfunction are prominent health care concerns. Despite increasing recognition of this emerging health care crisis, mechanisms accounting for rapid and progressive end-organ dysfunction following recovery from critical illness remains unknown.

Nosocomial pneumonia has been identified as inducing lung endothelial amyloid production. These amyloid species have been identified as cytotoxic, self-replicating, and transmissible. They have been described as insensitive to boiling, proteases, RNAse, and DNAse. Thus, infection-induced cytotoxic endothelial amyloids represent a form of prion disease, and emerging evidence has indicated that endothelial amyloids contribute to end-organ dysfunction in the aftermath of critical illness. However, mechanisms of host-pathogen interactions underlying production of endothelial amyloids has thus far remained unclear.

A need exists for new and effective antimicrobial compositions, including methods for production of such compositions and therapeutic methods associated with such compositions.

### BRIEF SUMMARY OF THE INVENTION

The current disclosure relates, at least in part, to the surprising and unexpected identification of cell-free amyloid-containing preparations that possess robust antimicrobial and biofilm-degrading and biofilm-inhibiting activities, which have initially been produced by, and isolated from, mammalian endothelial cells infected with infectious agents (e.g., bacteria) that either do not possess a Type 3 Secretion System (T3SS) and/or T3SS-related exoenzymes or are not capable of injecting T3 SS-mediated exoenzymes into the cytosol of the host cell. In certain embodiments, such infectious agents include Gram-negative bacteria possessing a T3SS having reduced or ablated T3SS activity. In other embodiments, supernatant produced by infecting endothelial cells with T3SS incompetent, Gram-positive bacteria, such as Staphylococcus aureus, has been found to elicit production and release of the same antimicrobial amyloids from Pulmonary Microvascular Endothelial Cells (PMVECs). It is further expressly contemplated that any infectious agent that either does not possess a T3SS and/or T3SS-related exoenzymes or is not capable of injecting T3SS-mediated exoenzymes into the cytosol of the host cell can elicit production of antimicrobial amyloids from PMVECs. In a prion-like manner, the effects of such amyloid-containing preparations upon mammalian endothelial cells are both transmissible and self-replicating (indeed, naïve mammalian endothelial cells appear to produce such antimicrobial amyloid compositions with even greater efficacy upon secondary and subsequent passages than primary, microbe-infected mammalian endothelial cells that provide initial supernatants, which are then used for further rounds of passage/propagation).

Accordingly, the instant disclosure provides at least the following: mammalian cell-derived antimicrobial amyloid preparations (which, in certain embodiments, are capable of biofilm degradation or inhibition or substantial attenuation of biofilm formation relative to a treatment without the inventive preparations/compositions); compositions and methods for production of such antimicrobial amyloid preparations; methods of treatment that employ antimicrobial amyloid preparations; and methods of treatment that rely upon amyloid oligomer immunodepletion/neutralization to treat infected cells, tissues, and/or an infected subject, with such methods appearing to act by reducing cytotoxicity of amyloid assemblies, which appear to be amyloid oligomer-containing complexes, in the immunodepletion therapy-treated cells, tissues, or subject and/or by enhancing the antimicrobial properties of amyloid assemblies that remain in treated cells or in a treated subject after amyloid oligomer immunodepletion.

In one aspect, the instant disclosure provides a method for producing and harvesting an antimicrobial amyloid protein composition, the method involving: (a) contacting a mammalian cell in cell culture media with an infectious agent that either does not possess a T3SS and/or T3SS-related exoenzymes or is not capable of injecting T3SS-mediated exoenzymes into the cytosol of the mammalian cell, thereby producing a first cell culture admixture; (b) incubating the first cell culture admixture for an amount of time sufficient to induce the mammalian cell production and release of an antimicrobial amyloid protein complex into the cell culture media; and (c) harvesting the cell culture media that harbors the antimicrobial amyloid protein complex, thereby producing and harvesting an antimicrobial amyloid protein composition.

In one embodiment, the mammalian cell is an endothelial cell or an epithelial cell (or a combination of both). Optionally, the mammalian cell is a PMVEC or an arterial endothelial cell (PAEC).

In certain embodiments, the infectious agent is a bacteria, optionally a Gram-positive bacterium or a Gram-negative bacterium that does not possess a T3SS and/or does not possess T3SS-related exoenzymes or is not capable of injecting T3SS-mediated exoenzymes into the cytosol of the mammalian cell, optionally a Gram-negative bacterium that does not possess a T3SS and/or does not possess T3SS-related exoenzymes or is not capable of injecting T3SS-mediated exoenzymes into the cytosol of the mammalian cell that is a Pseudomonas spp. bacteria or a Klebsiella pneumoniae bacterium. Optionally, the infectious agent is a Gram-negative bacterium possessing a T3SS having reduced T3SS activity and/or reduced T3SS-related exoenzyme activity. In a related embodiment, the bacteria possessing a T3SS having reduced T3SS activity and/or reduced T3SS-related exoenzyme activity is a Pseudomonas aeruginosa bacteria. In another embodiment, the bacteria possessing a T3SS having reduced T3SS activity and/or reduced T3SS-related exoenzyme activity is selected from the group consisting of a Pseudomonas aeruginosa ΔPcrV mutant, a Pseudomonas aeruginosa ΔUΔT mutant, Pseudomonas aeruginosa clinical isolate PA-35, and a Pseudomonas aeruginosa mutant possessing an ExoY cyclic nucleotidyl cyclase deficiency (which as used herein, embraces point mutations introduced to render ExoY antimicrobial such as a Pseudomonas aeruginosa possessing an ExoY^{K81M} point mutation).

In one embodiment, the antimicrobial amyloid protein complex of the harvested cell culture is non-cytotoxic and/or is capable of provoking biofilm degradation or inhibiting or substantially attenuating biofilm formation.

In another embodiment, step (b) includes incubating the first cell culture admixture for an initial period of time and refreshing the cell culture media after this initial period of time. Optionally, this initial period of time is about four hours, or is about five hours, or more.

In certain embodiments, the method further includes step (d) filter-sterilizing the cell culture that harbors the antimicrobial amyloid protein complex.

In another embodiment, the method further includes steps (e)-(g): (e) contacting a naïve mammalian cell with the harvested cell culture media that harbors the antimicrobial amyloid protein complex of step (c) and additional cell culture media, thereby producing a second cell culture admixture; (f) incubating the second cell culture admixture for an amount of time sufficient to induce in the naïve mammalian cell production and release of an antimicrobial amyloid protein complex into the cell culture media of the second cell culture admixture; and (g) harvesting the cell culture media of the second cell culture admixture that harbors the antimicrobial amyloid protein complex. Optionally, the method further involves repeating steps (e) through (g) two or more times (for a total of three or more passages), optionally between two and five times or more (for a total of 3-6 or more passages).

In another embodiment, the method further includes depletion or neutralization of tau amyloid species and depletion or neutralization of beta amyloid (Aβ) species. In certain embodiments, the method further includes sequential depletion or neutralization of tau amyloid species and depletion or neutralization of beta amyloid (Aβ) species, or sequential depletion or neutralization of Aβ species and then depletion or neutralization of tau amyloid species. Depletion or neutralization of these entities may be achieved by adding to the culture media/mixture an anti-tau antibody and an anti-Aβ antibody, both in amounts effective to deplete or neutralize these amyloid protein species. Yet another surprising and unexpected aspect of the present disclosure, as demonstrated in a working example herein, is that these additional steps further enhance the anti-microbial effect of the amyloid protein composition.

Another aspect of the instant disclosure provides an antimicrobial amyloid protein composition produced by a method of the instant disclosure.

An additional aspect of the instant disclosure provides a pharmaceutical composition that includes an antimicrobial amyloid protein composition produced by a method of the instant disclosure and a pharmaceutically acceptable carrier.

Another aspect of the instant disclosure provides a method for treating a subject having or at risk of developing a microbial infection that involves administering a pharmaceutical composition of the instant disclosure to the subject, thereby treating the subject having or at risk of developing a microbial infection.

In one embodiment, the microbial infection is a nosocomial infection. Optionally, the nosocomial infection is a nosocomial staphylococcus infection. Optionally, the nosocomial infection is a nosocomial pneumonia. Optionally, the microbial infection is antibiotic resistant and/or multi-drug resistant (MDR).

An additional aspect of the instant disclosure provides a method for treating or preventing a nosocomial infection, a systemic infection, a superficial infection and/or a burn in a subject in need thereof, the method involving administering to the subject a pharmaceutical composition of the instant disclosure, thereby treating or preventing a nosocomial infection, a systemic infection, a superficial infection and/or a burn in the subject. Optionally, the systemic infection is sepsis, meningitis and/or other such systemic infection. Optionally, the superficial infection is a superficial infection of the central nervous system (CNS) and/or other such superficial infection.

Another aspect of the instant disclosure provides a composition for producing an antimicrobial amyloid protein complex that includes a mammalian cell infected with: an infectious agent that does not possess a T3SS and/or that does not possess T3SS-related exoenzymes or an infectious agent that is not capable of injecting T3SS-mediated exoenzymes into the cytosol of the mammalian cell.

One aspect of the instant disclosure provides a method for treating a microbial infection in a subject, the method involving administering to the subject in need thereof an anti-amyloid antibody in an amount sufficient to deplete amyloid levels in the subject, thereby treating the microbial infection in a subject.

In one embodiment, the microbial infection is Pseudomonas aeruginosa bacteria, a Staphylococcus aureus bacteria and/or a Klebsiella pneumoniae bacteria. Optionally, the Pseudomonas aeruginosa bacteria possesses an intact T3SS.

In certain embodiments, the anti-amyloid antibody is capable of neutralizing or immunodepleting an amyloid or an amyloid oligomer in the subject. In a related embodiment, the anti-amyloid antibody is an anti-tau antibody, optionally an anti-tau oligomer antibody. Optionally the anti-tau antibody is a monoclonal anti-tau antibody, optionally a monoclonal anti-tau antibody that binds the oligomeric conformation of tau. Optionally, the anti-tau antibody is a monoclonal anti-tau T22 antibody, a monoclonal anti-tau TNT-1 or TNT-2 antibody or a monoclonal anti-tau TOC1 antibody. Optionally, the anti-amyloid antibody is an anti-Aβ antibody, a pan-anti-amyloid antibody or an anti-amyloid A antibody. Optionally, the anti-amyloid antibody is a conformationally specific pan-amyloid antibody, such as OC or the anti-amyloid antibody is a pan-amyloid antibody, such as A11. In a related embodiment, the anti-amyloid antibody is an anti-Aβ antibody specific for the oligomeric conformation of amyloid beta (Aβ). Optionally, the anti-Aβ antibody specific for the oligomeric conformation of Aβ is a monoclonal or a polyclonal anti-Aβ antibody specific for the oligomeric conformation of amyloid beta. Optionally, the monoclonal anti-amyloid beta antibody specific for unaggregated species and conformations of Aβ such as MOAB-2 and/or Aβ 1-40 antibody. Optionally, the polyclonal anti-Aβ antibody targeting any Aβ variant for Aβ is an Aβ 1-43 antibody.

In certain embodiments, the anti-amyloid antibody is administered in combination with an antimicrobial peptide treatment.

Another aspect of the instant disclosure provides a method for producing and collecting an antimicrobial amyloid protein composition, the method involving: (a) culturing a mammalian cell in a medium; (b) adding to the product of step (a) an infectious agent that does not possess a T3SS and/or does not possess T3SS-related exoenzymes or an infectious agent that is not capable of injecting T3SS-mediated exoenzymes into the cytosol of the mammalian cell, in an amount sufficient to induce the mammalian cell to produce and release amyloid protein complexes into the medium, thereby producing a medium product comprising amyloid protein complexes; (c) removing the medium product of step (b), thereby producing a mammalian cell product; (d) culturing the mammalian cell product of step (c) in a fresh medium for a time length sufficient to allow for mammalian cell production and release of amyloid protein complexes into the medium, thereby producing a second medium product comprising amyloid protein complexes; and (e) collecting the second medium product of (d), thereby producing and collecting an antimicrobial amyloid protein composition. Thus, following incubation of the original supernatant on a new naïve monolayer of cells e.g., for 4 hours, the initial admixture is removed, rinsed (e.g., 5× with HBSS), and fresh media is applied. Significantly, the original mixture is removed prior to the release of new amyloid constituents into the fresh media. Rather than being an accumulation, the new monolayer that is formed is stimulated to release a more pure, potent version without bacterial components involved. This sequence of steps can be repeated e.g., for 2, 3 or more passages which may result in a significant enhancement of antimicrobial effect.

In one embodiment, the method further includes step (f) recovering the amyloid protein complexes from the second medium product of step (e).

An additional aspect of the instant disclosure provides a composition that includes an antimicrobial amyloid protein composition produced by a method of the instant disclosure.

A further aspect of the instant disclosure provides a method for inhibiting or substantially attenuating formation of a biofilm on an in-dwelling catheter and/or endotracheal tube or degrading a biofilm present on an in-dwelling catheter and/or endotracheal tube, or a wound, the method involving application of an antimicrobial amyloid protein composition of the instant disclosure to the in-dwelling catheter and/or endotracheal tube. Optionally, the in-dwelling catheter and/or endotracheal tube resides in a mammalian subject.

### DETAILED DESCRIPTION OF THE INVENTION

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. In some embodiments, administration is oral. Additionally, or alternatively, in some embodiments, administration is parenteral. In some embodiments, administration is intravenous.

The term “biofilm” as used herein refers to a material which naturally develops when microbes attach to a support that is made of a material including but not limited to teeth, mucous membranes, soft tissue surfaces, medical implants, stone, metal, plastic, glass and wood. “Biofilm” also refers to filamentous and non-filamentous bacteria that produce an extracellular polysaccharide, extracellular DNA, and proteinaceous matrix that act as a natural glue to immobilize and protect the bacterial cells from the environment and/or the host's immune response. In certain embodiments, the term “biofilm” as used herein refers to substances that contain either single or multiple microbial species and that readily adhere to such diverse surfaces as river rocks, soil pipelines, teeth, mucous membranes, and medical implants. Biofilms are biological films that can develop and persist on solid substrates in contact with moisture, on soft tissue surfaces in living organisms and at liquid air interfaces. They can develop into structures several millimeters or centimeters in thickness and can cover a large surface area. In nature, non-filament-forming microorganisms stick to the biofilm surface, locating within an area of the biofilm that provides an optimal growth environment with respect to pH, eH, dissolved oxygen, and nutrients. Since nutrients tend to concentrate on solid surfaces, including porous surfaces and wet, dry surfaces, a microorganism saves energy through cell adhesion to a solid surface rather than by growing unattached. Microbes are capable of attachment to almost any surface submerged in an aqueous environment—a phenomenon known as microbial adhesion. Colonization and proliferation of the microbes on a surface forms a biofilm. Adhesion of microbes on a surface is involved in diseases of humans and animals, in dental plaque formation, in industrial processes, in fouling of man-made surfaces, in syntrophic and other community interactions between microorganisms, and in the activity and survival of microorganisms in natural habitats. Biofilms are particularly noted as largely impervious to antibiotic treatment and are the fundamental means by which the chronic Pseudomonas infection-mediated poor outcomes and high healthcare costs associated with cystic fibrosis patients are facilitated. Biofilms also often contain extracellular DNA and bacterial amyloids.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects—commonly for the instant disclosure, a vehicle control (e.g., HBSS) is employed as a control. Methods to select and test control samples are within the ability of those in the art. Determination of statistical significance is within the ability of those skilled in the art, e.g., the number of standard deviations from the mean that constitute a positive result.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference.). A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A and 1B demonstrate that filter-sterilized supernatants obtained from pulmonary microvascular endothelial cells (PMVECs) infected with P. aeruginosa strain PA01 exhibited a cytotoxic effect upon naïve PMVECs, in a transmissible manner indicative of a prion-type of mode of action. FIG. 1A shows infected source PMVEC cells and the initial transfer of filter-sterilized, bacteria-free infection-derived supernatant to naïve PMVECs at TO. FIG. 1B shows the cytotoxic impact upon such cells of a 16-hour incubation with the cellular supernatant of the initially infected PMVECs.

FIG. 2 illustrates that the cytotoxic effect observed in FIGS. 1A and 1B was self-replicating.

FIG. 3 illustrates the generation of the primary supernatant via the infection of PMVECs with bacteria.

FIG. 4 shows an image, for a population of Hank's Balanced Salt Solution (HBSS)-treated cells (which constitute a vehicle control, bacterial infections of PMVECs were performed in HBSS. Initial infected culture supernatants were then filter-sterilized and thereby rendered bacteria-free, leaving the amyloid entities suspended in HBSS. Therefore, HBSS was employed that had gone through the entire process sans bacteria (i.e., PMVECs were treated with HBSS just as if they were going to receive bacteria, but no bacteria were added. The cells were then incubated with the HBSS for the length of the infection, collected at the same time as bacterially infected cells, and the Vehicle HBSS control was then centrifuged and filter-sterilized just as if it had bacteria. The resulting suspension was the ‘HBSS’ control, which was also more commonly referred to as ‘vehicle control’, which demonstrated the (lack of) impact of primary supernatant infection at 16 hours.

FIG. 5 shows an image, for a population of P. aeruginosa strain PA103 (wt) cells, that demonstrates the impact of primary supernatant treatment on naïve PMVECs at 16 hours. Arrowheads indicate regions of particular impact—specifically, interendothelial cell gaps. The P. aeruginosa strain PA103 was thereby observed to produce a less cytotoxic supernatant when infecting PMVECs than P. aeruginosa strain PA01.

FIG. 6 shows an image, for a population of endothelial cells infected with the P. aeruginosa ΔPcrV (which possesses a non-functional T3SS, as it is a mutant of strain PA103. PcrV is the shelf protein in the tip of the T3SS—in this mutant, the PcrV element has been knocked-out and the bacterium is unable to inject the T3SS exoenzymes into the interior of the host cell), that demonstrates the relative absence of cytotoxic impact of primary supernatant treatment at 16 hours, in contrast to the cytotoxic effects observed in, e.g., FIGS. 1B and 7, which were infected with P. aeruginosa strains possessing functional T3SSs.

FIG. 7 shows an image, for a population of P. aeruginosa strain PA01 cells, that demonstrates the cytotoxic impact of primary supernatant treatment at 16 hours.

FIG. 8 shows an image, for a population of P. aeruginosa strain PA01 cells in which amyloid oligomers were immunodepleted, that demonstrates the relative absence of cytotoxic impact of primary supernatant treatment at 16 hours (as compared, e.g., to the cells of FIGS. 1B and 7).

FIG. 9 illustrates that cytotoxicity exists on a continuum.

FIG. 10 illustrates that antimicrobicity also exists on a continuum (notably, infection of PMVECs, particularly by a T3SS-mutated ΔPcrV P. aeruginosa derived from strain PA103 (P. aeruginosa ΔPcrV) induced the release of antimicrobial amyloids from PMVECs).

FIGS. 11A and 11B demonstrate implementation of a Kirby-Bauer disk diffusion assay, commonly used for antibiotic sensitivity testing of bacterial isolates. FIG. 11A shows that different antibiotics produced varying zone of inhibition (halo) sizes. FIG. 11B shows results for negative control (HBSS), positive control (gentamicin) and a supernatant preparation from PMVECs infected with the T3SS-mutant P. aeruginosa ΔPcrV, in the Kirby-Bauer disk diffusion assay.

FIG. 12 shows a graph demonstrating that P. aeruginosa ΔPcrV infection of PMVECs induced the release of antimicrobial amyloids, whereas T3SS effector intoxication (as observed for T3SS intact P. aeruginosa strain PA103-infected PMVECs) suppressed antimicrobial activity of endothelial amyloids. 1-way ANOVA with Neuman-Keuls post-hoc; p<0.05.

FIG. 13 shows a graph demonstrating a time course of the antimicrobial effect observed for P. aeruginosa ΔPcrV infection of PMVECs. The graph shows levels of ΔPcrV antimicrobial inhibition of Pseudomonas spp. at the indicated time points. n=18; Kruskall-Wallis with Dunn's post-hoc; p<0.001.

FIG. 14 shows that PMVEC supernatant obtained from cells infected with an ExoY mutant of P. aeruginosa was observed to have reduced cytotoxic activity when applied to naïve PMVECs.

FIG. 15 shows that PMVEC supernatant obtained from cells infected with ExoY mutant ExoY^{K81M} (possessing a catalytically inactive ExoY, with a functional T3SS but non-functional effector) of P. aeruginosa was observed to have reduced cytotoxic activity when applied to naïve PMVECs.

FIG. 16 shows that PMVEC supernatant obtained from cells infected with P. aeruginosa strain PA01, which was then immunodepleted for tau amyloid oligomers, exhibited reduced cytotoxic activity when applied to naïve PMVECs.

FIG. 17 shows that immunodepletion of T3SS-induced amyloid oligomers rescued the antimicrobial activity of endothelial amyloids, post-infection. n=5; Kruskall-Wallis with Dunn's post-hoc; *p<0.05.

FIG. 18 shows that although endothelial amyloids produced from the infection of PMVECs with T3SS-competent P. aeruginosa are not antimicrobial (e.g., PA103 SN, PAO1 SN), the neutralization of T22-reactive tau oligomers from these admixtures rescues antimicrobial activity equivalent with that of amyloids derived from the infection of PMVECs with T3 SS-deficient ΔPcrV.

FIGS. 19A and 19B shows P. aeruginosa strain PA01 biofilm produced on YESCA Congo Red minimal media. FIG. 19A shows uninoculated cells viewed at 4×. FIG. 19B shows the uninoculated amyloid-rich biofilm of PA01 viewed at 10×.

FIGS. 20A and 20B show that the amyloid-rich biofilm of the P. aeruginosa strain PA01 on YESCA Congo Red agar was broken down via administration of endothelial amyloids; FIGS. 20A and 20B show two respective fields of treated cells viewed at 10×.

FIGS. 21A to 21F show the effect of different supernatants upon established lawns of the P. aeruginosa strain PA01. FIG. 21A shows a lawn of PA01 (at 37° C.). FIG. 21B shows the effect of Gentamicin in attenuating the PA01 lawn. FIG. 21C shows the lawn after application of HBSS as a negative control. FIG. 21D shows the biofilm lawn after application of T3SS-competent mutant ExoY⁺ supernatant. FIG. 21E shows increased aggregation of bacteria from the PA01 lawn after application of non-tau amyloid-depleted ExoY⁺ was applied. FIG. 21F shows augmented bacterial punctate aggregation after application of tau amyloid-depleted supernatant of ExoY+ was applied.

FIGS. 22A to 22C show results of studies that queried whether other infected cell types exhibited bacteriostatic activity. FIG. 22A shows Kirby-Bauer disk diffusion assay results for gentamicin-treated, HBSS (negative control)-treated, ΔPcrV supernatant (PcrV SN)-treated, CSF-treated, BALF-treated and PA103 supernatant-treated lawns. FIG. 22B shows a graph of zone of inhibition results for the gentamicin-treated, HBSS (negative control)-treated, ΔPcrV supernatant (PcrV SN)-treated, CSF-treated and PA103 supernatant-treated assays (n=6, one-way ANOVA with Neuman-Keuls post-hoc; ***p<0.001). FIG. 22C shows a graph of zone of inhibition results obtained for the gentamicin-treated, HBSS (negative control)-treated, ΔPcrV supernatant (PcrV SN)-treated, BALF-treated and PA103 supernatant-treated assays (n14, one-way ANOVA with Neuman-Keuls post-hoc; ***p<0.001).

FIG. 23 shows tabulation of the positive inhibition results observed for gentamicin-treated, HBSS (negative control)-treated, ΔPcrV supernatant (PcrV SN)-treated, BALF-treated, CSF-treated and PA103 supernatant-treated assays in the graphed assays of FIGS. 22B and 22C.

FIG. 24 shows aggregates at 40× magnification.

FIG. 25 shows the effects observed for an HBSS (negative control)-treated PA103 lawn, at 10× magnification.

FIGS. 26A and 26B show examples of advancing inhibition observed for a ΔPcrV supernatant (PcrV SN)-treated PA103 lawn, at 10× magnification. FIG. 26A shows a front of advancing inhibition observed for a ΔPcrV supernatant (PcrV SN)-treated PA103 lawn, at 10× magnification. FIG. 26B shows another example of advancing inhibition observed for a ΔPcrV supernatant (PcrV SN)-treated PA103 lawn, at 10× magnification.

FIGS. 27A and 27B demonstrate time-dependent observed anti-microbial activity of supernatants upon Pseudomonas spp. FIG. 27A shows the progression of ΔPcrV supernatant-mediated inhibition of Pseudomonas spp. (n18; Kruskall-Wallis with Dunn's post-hoc; ***p<0.001). FIG. 27B shows the progression of PA103 supernatant-mediated inhibition of Pseudomonas spp. (n=7; Kruskall-Wallis with Dunn's post-hoc; p*<0.01).

FIG. 28 demonstrates time-dependent observed anti-microbial activity of ExoY^{K81M} supernatant upon Pseudomonas spp.

FIG. 29 demonstrates observed anti-microbial activity of ExoY supernatant upon Pseudomonas spp., as compared to gentamicin, HBSS (negative control), PA01 supernatant and ExoY^{K81M} supernatant.

FIG. 30 is a graph that shows antimicrobial aggregation (as a % of total area) of established lawns of bacteria following treatment with aliquots of bacteria-free amyloid suspensions obtained from endothelial cells infected with T3SS-deficient ΔPcrV, wherein the aliquots of ΔPcrV supernatant were then either neutralized with a single anti-amyloid antibody, or sequentially with more than one anti-amyloid antibody in a serial neutralization.

FIGS. 31A-D are two pairs of corresponding photographs and graphs that show substantial attenuation of biofilm formation by endothelial amyloids in a standard microtiter plate crystal violet biofilm assay using round-bottomed polyvinyl chloride (PVC) plates to replicate endotracheal tube and in-dwelling catheter material and design. Images show the thick biofilm, or pellicle, formed at the air-liquid interface by the ExoY-competent nosocomial pneumonia isolate PA-815 (FIG. 31A) and the lab strain PA01 (FIG. 31C). FIGS. 31B and 31D demonstrate that T3SS-deficient ΔPcrV infection-derived supernatant significantly attenuated biofilm formation on PVC substrate, proving particularly effective in the prophylaxis of clinical isolate PA-815 generated biofilm as compared to the negative control. Moreover, subsequent generations of passaged amyloids (2° ΔPcrV and 3° PcrV) exhibited equivalent efficacy. n=10; 5 technical replicates for each independent experiment; mean±SEM; one-way ANOVA with Dunnett's post-hoc. *p<0.01, **p<0.001, ***p<0.0001.

### DETAILED DESCRIPTION OF THE INVENTION

The present disclosure is directed, at least in part, to the discovery that cells infected by an infectious agent that either does not possess a T3SS and/or does not possess T3SS-related exoenzymes or is not capable of injecting T3SS-mediated exoenzymes into the cytosol of a mammalian cell produce endothelial amyloids that exhibit broad-spectrum antimicrobial activity. These endothelial amyloids can be collected and purified from the cell culture media for use as an antimicrobial. It was further identified that antimicrobial amyloid preparations could be obtained from endothelial cells infected with Gram-negative bacteria possessing mutant T3SS-associated molecules or lacking T3SS-associated exoenzymes, or via amyloid assembly and/or amyloid oligomer immunodepletion of endothelial cells infected with microbes that possess an intact T3SS. Antimicrobial preparations, infected cell-based platforms for antimicrobial preparation production, and methods for treatment of subjects having microbial infections (whether via administration of antimicrobial preparations of the instant disclosure or via administration of antibodies specific for immunodepletion of amyloid oligomers) are therefore provided.

Previously, infection-induced amyloids have been identified to contribute to insidious end-organ dysfunction that leads to unacceptably high morbidity, mortality and health care cost following critical illness. However, as newly demonstrated herein, the endothelial amyloids of the instant disclosure exhibited antimicrobial and cytotoxic properties that were unexpected in terms of their scope and efficacy (e.g., certain of the instant supernatant preparations showed efficacy against biofilms, which is at least one effect distinguishable from prior descriptions of antimicrobial properties attributed to, e.g., synthetic Aβ peptides in isolation). Indeed, the instant disclosure provides evidence that specific and adjustable forms of infection of lung endothelial cells were capable of eliciting endothelial amyloids with distinguishable antimicrobial and cytotoxic properties.

Historically, amyloids have been regarded as pathological markers of chronic disease, including neurodegenerative diseases such as Alzheimer's, Parkinson's, and transmissible spongiform encephalopathies (www.brightfocus.org/alzheimers/infographic/amyloid-plaques-and-neurofibrillary-tangles). However, it has been established herein that Pseudomonas aeruginosa infection elicits the acute production of cytotoxic endothelial amyloids that are transmissible, self-replicating, and resistant to DNAse, RNAse, and proteases. Cytotoxic amyloid species arise in response to T3 SS exoenzyme intoxication following P. aeruginosa infection. In the absence of T3 SS effector modulation, pulmonary endothelial amyloid species have newly been described herein to effect significant bacteriostasis secondary to bacterial challenge, as assessed by standard Kirby-Bauer disk diffusion assay and direct inoculation of bacterial lawns. The instant disclosure has therefore also described selection of antimicrobial amyloid species that exhibit broad-spectrum antimicrobial and antibiotic activity and that inhibit common nosocomial pathogens, including Staphylococcus aureus, Klebsiella pneumoniae, and P. aeruginosa strains in both a dose- and time-dependent manner. Provocatively, these antimicrobial amyloid species have also been demonstrated herein to be highly effective in breaking down the robust amyloid-rich biofilm of the P. aeruginosa strain PA01. The instant disclosure therefore provides for harvesting—and optional enrichment—of novel antimicrobial compounds as therapeutics for antibiotic resistant organisms. This therapeutic option is likely to provide an additional line of treatment for nosocomial pneumonia and has the potential to abrogate the increased rates of end-organ dysfunction, neurocognitive decline, and early mortality seen among critically ill patients recovering from nosocomial pneumonia post-discharge. Additional explicitly contemplated applications for antimicrobial endothelial amyloids include the treatment of burns, wounds, sepsis, cystic fibrosis, and infection. In particular, the endothelial amyloid species described herein provide a promising burn wound treatment in light of the demonstrated efficacy of such endothelial amyloid species against Pseudomonad species. The ability of these compounds to aggressively degrade biofilms also makes them suitable for the treatment of in-dwelling catheters and endotracheal tubes.

The antimicrobial preparations of the instant disclosure are therefore provided as therapeutics for antibiotic resistant organisms that infect both humans and animals, among other compositions and methods as set forth herein. The ability of these antimicrobial amyloid preparations to aggressively degrade biofilms or inhibit or substantially attenuate or retard their formation is also explicitly contemplated for the treatment of in-dwelling catheters and endotracheal tubes.

Compositions and preparations of antimicrobial amyloids, as well as methods for production and use of such antimicrobial amyloids are described in additional detail below. Immunodepletion approaches for eliminating cytotoxic properties of amyloid oligomers, including therapeutic application of such approaches, are also described in additional detail below.

### Amyloid Proteins and Complexes

Amyloid proteins are defined by their quaternary structure, which includes a ‘cross-beta structure’. Many amyloids are aggregates of proteins that become folded into a shape that allows many copies of that protein to stick together, forming fibrils. In the human body, amyloids have been linked to the development of various diseases. Pathogenic amyloids form when previously healthy proteins lose their normal physiological functions and form fibrous deposits in plaques around cells which can disrupt the healthy function of tissues and organs. It is noted that some beta sheet-rich proteins with an intrinsically disordered domain can assume an amyloid conformation (e.g., the amyloid fold) but many are structurally sound and physiologically functional in various conformers e.g., monomeric form (tau), meaning that not all amyloids form fibrils.

Specific peptides known to form amyloids, including amyloid oligomers, include amyloid p and tau peptides, among other amyloid-forming peptides. For the antimicrobial amyloid-containing complexes (amyloid assemblies) of the present disclosure, it is explicitly contemplated that S100 amyloid proteins and alpha synuclein may well comprise a component(s) of these antimicrobial complexes.

Amyloids have been associated with (but not necessarily as the cause of) more than 50 (Knowles et al. 2014. Nature Reviews Molecular Cell Biology. 15: 384-396) human diseases, known as amyloidosis, and may play a role in some neurodegenerative disorders (Pulawski et al. 2012. Applied Biochemistry and Biotechnology. 166: 1626-43). Some amyloid proteins are infectious, and certain forms of amyloids can be characterized as prions, in which the infectious form can act as a template to convert other non-infectious proteins into infectious form (Soto et al. 2006. Trends in Biochemical Sciences. 31 (3): 150-5). Amyloids may also have normal biological functions; for example, in the formation of fimbriae in some genera of bacteria, transmission of epigenetic traits in fungi, as well as pigment deposition and hormone release in humans (Toyama and Weissman. 2011. Annual Review of Biochemistry. 80: 557-85).

Amyloids have been known to arise from many different proteins and polypeptides (Ramirez-Alvarado et al. 2000. PNAS. 97: 8979-84). These polypeptide chains generally form 0-sheet structures that aggregate into long fibers; however, identical polypeptides can fold into multiple distinct amyloid conformations. The diversity of the conformations may have led to different forms of the prion diseases.

The classical, histopathological definition of amyloid is an extracellular, proteinaceous deposit exhibiting beta sheet structure. Common to most cross-beta-type structures, in general, they are identified by apple-green birefringence when stained with Congo red and seen under polarized light. These deposits often recruit various sugars and other components such as Serum Amyloid P component, resulting in complex, and sometimes inhomogeneous structures (Sipe and Cohen. J. 2000. Struct. Biol. 130: 88-98). Recently this definition has come into question as some classic, amyloid species have been observed in distinctly intracellular locations (Lin et al. 2007. Diabetes. 56: 1324-32).

A more recent, biophysical definition is broader, including any polypeptide that polymerizes to form a cross-beta structure, in vivo or in vitro. Some of these, although demonstrably cross-beta sheet, do not show some classic histopathological characteristics such as the Congo red birefringence. Microbiologists and biophysicists have largely adopted this definition (Nilsson. 2004. Methods (San Diego, Calif.). 34: 151-60; Fandrich. 2007. CMLS. 64: 2066-78).

### Antimicrobial Amyloids

Amyloid species are heterogeneous in nature, and they include both cytoprotective and cytotoxic “strains”. In centrifugation experiments it was previously noted that short-term high-speed centrifugation removed an apparent cytoprotective factor(s). Immunoblotting revealed that this centrifugation step pelleted large protein bands, or complexes, recognized by both A11 (anti-Aβ oligomer) and T22 (anti-Tau oligomer) antibodies, but did not remove lower bands that had previously been shown to be cytotoxic. Determination of the molecular basis of the cytoprotective factor(s) was sought, as such factor(s) could serve as a potential therapeutic. An Aβ antibody was initially identified that, like short-term centrifugation studies, neutralized cytoprotective factor(s). Elution of this factor from the antibody resulted in significant cytoprotection against amyloid-induced injury. Thus, the instant factor is currently being purified, concentrated, and then tested as a novel therapy for treatment during infection. The studies disclosed herein have supported the concept that multiple amyloid species are produced, including some that are cytotoxic to mammalian cells and some that are not. In the latter case, an Aβ species produced by endothelium has been identified herein as at least a component of an amyloid assembly that is cytoprotective.

How endothelial-derived amyloids contribute to cytoprotection has been examined herein, as well as whether they may also fulfill an essential role in innate immunity. Previously, Soscia et al. had reported a direct interaction between amyloids, principally recombinant Aβ oligomers, and fungal and bacterial species, including P. aeruginosa (2010. PLoS One. 5(3): e9505; Kumar et al. 2016. Sci Transl Med. 8(340): 340ra72). Later, Eimer et al. described the interaction of endogenous neuronal Aβ with viral species (2018. Neuron. 99(1): 56-63.e3). Aβ binding to the microbial cell wall initiated protofibril formation that captured the organisms. The resulting fibrillar Aβ-bacterial interaction was bactericidal.

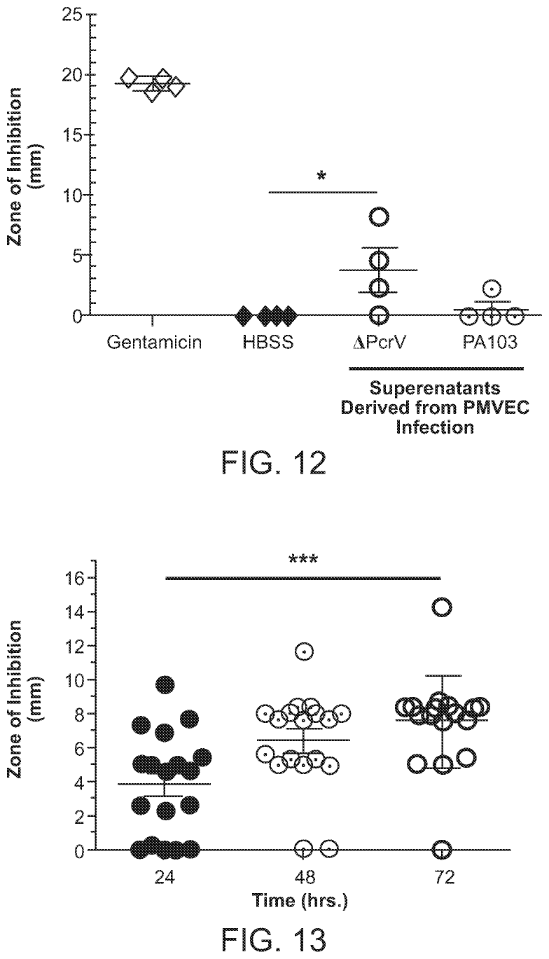

In certain aspects of the instant disclosure, whether cytotoxic amyloids generated by alveolar-capillary endothelial cells might possess antimicrobial properties was examined. Bacteria lacking either a functional type 3 secretion system (e.g., ΔPcrV) or functional type 3 secretion system effectors (e.g., Exo^{YK81M}) were herein identified as eliciting production of endothelial amyloids with no cytotoxic activity. However, these amyloids possessed very significant bacteriostatic and bacteriocidal activity (FIGS. 111B, 12, 13, 18, 22A-22C, 23, 27A, 27B, 28 and 29). ΔPcrV infected cells generated supernatant possessing the highest antimicrobial activity, followed in rank order by endothelium infected with ExoY^{K81M}, PA103, ExoY⁺ and PA01. These results were then compared to supernatant cytotoxicity (FIG. 10), and an inverse relationship was discovered. In this case, T3SS-competent PA01 and ExoY⁺ infections elicited endothelial supernatant with high cytotoxicity, but with virtually no antimicrobial activity. Thus, these data revealed that the type 3 secretion system, and its effectors, acted to convert antimicrobial amyloids into cytotoxic amyloids that possess essentially no antimicrobial activity. These findings of the instant disclosure illustrate a critically important and previously unrecognized host-pathogen interaction.

It has further been examined herein whether oligomerized tau and Aβ might contribute to the balance between cytotoxicity and antimicrobial activity (FIG. 10). T22 has been identified to neutralize, i.e., reduce, cytotoxicity of the supernatant generated by PA103, ExoY⁺ and PA01 infections. However, in antimicrobial studies, T22 neutralization increased the antimicrobial properties of supernatant obtained from ΔPcrV infections. In contrast, A11 and A042 neutralizing antibodies have been identified herein as eliminating antimicrobial properties of the supernatant. These exciting data have revealed that type 3 secretion system effectors, especially ExoY, elicit production and release of oligomerized tau, which is mechanistically responsible for reducing antimicrobial activity and increasing cytotoxicity of the supernatant, whereas supernatant A042 kills bacteria.

The findings of the instant disclosure support the idea that infection leads to production of lung-derived amyloids possessing antimicrobial and/or cytotoxic activity. This observation was initially made in lung endothelial cells and has now been expanded to include alveolar epithelial cells, with certain differences noted regarding this host-pathogen mechanism. These studies have indicated that lung alveolar-capillary cells possess the ability to generate amyloids possessing antimicrobial activity as part of the innate immune defense mechanism, and further, that bacteria have evolved mechanisms capable of eliminating the antimicrobial properties of these amyloids and converting them into species cytotoxic to the host. Harnessing the antimicrobial nature of these amyloids, as disclosed herein, is specifically identified as a novel add-on therapy for use with antibiotics, since these amyloids are not only capable of killing bacteria, but also breaking down biofilms or substantially attenuating biofilm formation. Targeting cytotoxic amyloids can also serve as a novel therapy during infection, and in the aftermath of critical illness, since these amyloids are transmissible and self-replicating, and may contribute significantly to end-organ dysfunction.

### Amyloid Biology

The idea that proteins fold into three-dimensional structures that are necessary for their appropriate function is a foundational principle in biochemistry. Most teaching is based on Anfinsen's principle, that a single amino acid sequence gives rise to a single three-dimensional structure. However, it is increasingly clear that this principle does not apply to all proteins. Proteins that represent exceptions to this rule are referred to as intrinsically disordered proteins. Intrinsically disordered proteins can acquire interconverting structures. Some of these structures include protein aggregates. Protein aggregates can be either functional or dysfunctional. Examples of functional protein aggregates include the curli poteins generated by enteric bacteria. Curli proteins promote surface adhesion and biofilm formation. Another example is the neuronal cytoplasmic polyadenylation element binding protein, which induces mRNA translation necessary for formulating long-term memory. Examples of dysfunctional protein aggregates include tau and Aβ soluble oligomers, aggregates and/or filaments. These latter cases represent amyloid proteins with potentially catastrophic clinical consequences.

Amyloids are broadly defined as a “starch-like protein deposits in tissues.” They have characteristic β-sheet conformations. A variety of mechanisms contribute to the formation of diverse secondary structures, including phosphorylation and/or glycation. These proteins form soluble oligomers that can further fold into aggregates and complex filaments (amyloid oligomers, in particular, are cytotoxic). The mechanisms responsible for transitions between the various protein forms are complex, incompletely understood, and the focus of intensive study in the field. What is clear, however, is that various amyloid conformers, also referred to as “strains” or “species,” have heterogeneous functions. For example, some amyloid species appear innocuous or cytoprotective, whereas others are intensely cytotoxic. This concept is true in the instant studies, as endothelial amyloids can have antimicrobial or cytotoxic properties, depending upon the strain of bacteria they are exposed to. Certain aspects of the instant disclosure have described a novel mechanism of amyloid generation at the alveolar-capillary interface in response to infection. A unique opportunity was thereby presented to test the mechanism of amyloid production and function during the natural history of disease, which has never before been accomplished.

Infection elicits the production and release of complex, albeit still incompletely described, amyloids from alveolar-capillary cells. Tau and Aβ are among the amyloids liberated in this setting. Both tau and Aβ have previously garnered considerable attention because of their relationship to Alzheimer's Disease and other related dementias. Tau and Aβ are not the only proteins that are mechanistically linked to dementias, but in virtually all cases to date, the causative mechanism is thought to be a dysfunctional amyloid structure. These proteins seem to be self-replicating and transmissible, and thus share common features of prion disease.

In rare instances, genetic mutations in key genes responsible for amyloid production account for early onset dementia. For example, presenilin-1 is a component of gamma secretase, and mutations in presenelin-1 cause early onset Alzheimer's Disease. However, mutations like this account for less than 10% of the known dementia cases. Apolipoprotein E-e4 mutations are risk factors for late onset Alzheimer's Disease, but no specific gene has been identified that is responsible for late onset disease. The instant studies have not involved Alzheimer's Disease or chronic dementia per se. However, compelling evidence has been provided herein that nosocomial pneumonia elicits production of amyloids from the distal lung that are capable of distributing through the circulation to contribute to end-organ dysfunction. Behavioral and electrophysiological evidence that information processing in the hippocampus is impaired by these infection-elicited amyloids has also been provided herein. Indeed, lung-derived amyloids appear to contribute to memory deficits in the critical care setting. The mechanisms of this response are currently being dissected in detail, and the scope of the current studies is being expanded to rigorously test the prevalence of this phenomenon in intensive care unit patients. Evidence that bacteria elicit production of amyloids that impair memory and learning indicate that this mechanism may initiate cellular responses contributing to various forms of chronic dementias.

### Therapeutic Amyloid Protein Assembly Production

In certain embodiments, for production of the antimicrobial amyloid protein assembly compositions of the instant disclosure, the following parameters are specifically contemplated:
Exemplary mammalian cells used in the preparation of antimicrobial amyloid protein assembly compositions of the instant disclosure can be, e.g., endothelial cells, for which the greatest amount of supporting data has been produced to date (e.g., specifically Pulmonary Microvascular Endothelial Cells (PMVECs) or pulmonary arterial endothelial cells (PAECs). However, it is specifically contemplated that the instant production approaches are likely to work in epithelial cells, as well.
To produce the non-cytotoxic, anti-microbial, biofilm-degrading amyloid complexes of the instant disclosure, a key feature of the bacteria appears to be that they lack a T3SS or are T3SS-incompetent/compromised (e.g., such as certain forms of Gram-negative bacteria as known in the art and/or as described herein). Although the instant disclosure presents the most data for Pseudomonas aeruginosa, other bacteria meeting the same criteria are also explicitly contemplated for production of the antimicrobial amyloid preparations described herein—without wishing to be bound by theory, so long as such microbes cannot directly inject exoenzymes (e.g., exotoxin) into the mammalian cell.
Notably, if the harvested amyloid product of the initial mammalian cell-bacteria incubation process is further passaged through two or more additional iterations as described herein (optionally 3-5 or more additional iterations), the potency and effectiveness of the subsequent antimicrobial amyloid products is greatly improved. Without wishing to be bound by theory, this is attributed to the “prion-like”, self-propagating features of the amyloid complex.

### Amyloid Protein Assembly Characteristics

Notable characteristics of the antimicrobial amyloid complexes of the instant disclosure appear to be the following:
The antimicrobial amyloid complexes of the instant disclosure appear to consist predominately of Aβ and tau peptides, but other amyloids may be present in the instant preparations.
The ratio of specific amyloid peptides within the instant antimicrobial amyloid preparations is currently being examined.
Without wishing to be bound by theory, there are post-translational modifications to which the unique activity of the instant antimicrobial amyloid preparations can be attributed; however, the exact identity of such modifications is being investigated. Hyper-phosphorylation is likely a factor. It has been observed that antimicrobial activity of the instant preparations can be lost via incubation with phosphatase inhibitors. Without wishing to be bound by theory, hyperphosphorylation may inhibit activity (e.g. it may be a way of preventing a hyper-immune response) and the phosphatases dephosphorylate the amyloid complex to activate the antimicrobial activity.
The instant antimicrobial amyloid preparations have been characterized for: mass (via Aβ antibody and tau antibodies)—between 5-200 kDa with some smears on blots that are larger, up to around 250 kDa; Activity—(i) broad-spectrum antimicrobial properties (bacterio-static and -cidal activity is time-dependent, which is important); and (ii) ability to degrade and prevent formation of biofilms; Aggregation—the preparations have exhibited the ability to effectively aggregate microbes in a time-dependent manner; Self-propagating—without wishing to be bound by theory, the instant antimicrobial amyloid preparations can self-propagate in mammalian cells, similar to prions; The more the amyloid complex preparations are passaged in mammalian cells, allowing for self-propagation to work, an increase has been observed in all above-listed attributes; The activities of the antimicrobial amyloid preparations have also been identified herein as heat stable.

### Infectious Agents

The instant disclosure expressly contemplates that any microbe that does not have a fully functional T3SS capable of injecting T3SS-mediated exoenzymes—expressed from the chromosome or from a plasmid—into the host cell, is capable of inducing the release of the antimicrobial amyloid complexes of the instant disclosure to varying degrees. The greater the degree of T3SS dysfunction (e.g., mutation of the translocon complex), the greater the yield of antimicrobials.

In particular, the lack of T3SS competency (including mutants with T3SS exoenzymes deleted from their genome but retaining a functional needle complex) has been observed herein to drive amyloid complexes towards antimicrobial efficacy. Notably, there are many proteins/exoenzymes in the T3SS that can be mutated—e.g., ΔUΔT (exoenzymes deleted but functional needle) has also been identified herein to elicit antimicrobial amyloids. T3SS is restricted to Gram negative bacteria (is a marker of high virulence) and is expressed by: Yersinia spp. (plague and gastroenteritis), E. coli (virulent spp. EHEC, EPEC), Shigella spp, Salmonella spp., Vibrio spp. (gastroenteritis, cholera, necrotizing fasciitis), Chlamydia spp. (STD and Pneumonia), Burkholderia spp. (Malleodosis, nosocomial infections, gastroenteritis), Bordetella spp. (whooping cough, respiratory infections).

### Gram-Negative Bacteria

Gram-negative bacteria are bacteria that do not retain the crystal violet stain used in the gram-staining method of bacterial differentiation (Baron et al. Baron's Medical Microbiology (4th ed.). Univ of Texas Medical Branch). They are characterized by their cell envelopes, which are composed of a thin peptidoglycan cell wall sandwiched between an inner cytoplasmic cell membrane and a bacterial outer membrane.

Gram-negative bacteria are found everywhere, in virtually all environments on Earth that support life. The Gram-negative bacteria include the model organism Escherichia coli, as well as many pathogenic bacteria, such as Pseudomonas aeruginosa, Neisseria gonorrhoeae, Chlamydia trachomatis, and Yersinia pestis. They are an important medical challenge, as their outer membrane protects them from many antibiotics (including penicillin); detergents that would normally damage the peptidoglycans of the (inner) cell membrane; and lysozyme, an antimicrobial enzyme produced by animals that forms part of the innate immune system. Additionally, the outer leaflet of this membrane comprises a complex lipopolysaccharide (LPS) whose lipid A component can cause a toxic reaction when these bacteria are lysed by immune cells. This toxic reaction can include fever, an increased respiratory rate, and low blood pressure a life-threatening condition known as septic shock (Pellitier LL Jr, “Microbiology of the Circulatory System” “NCBI Bookshelf”).

Several classes of antibiotics have been designed to target Gram-negative bacteria, including aminopenicillins, ureidopenicillins, cephalosporins, beta-lactam-betalactamase combinations (e.g. pipercillin-tazobactam), Folate antagonists, quinolones, and carbapenems. Many of these antibiotics also cover Gram-positive organisms. The drugs that specifically target Gram-negative organisms include aminoglycosides, monobactams (aztreonam) and Ciprofloxacin.

The proteobacteria are a major phylum of Gram-negative bacteria, including Escherichia coli (E. coli), Salmonella, Shigella, and other Enterobacteriaceae, Pseudomonas, Moraxella, Helicobacter, Stenotrophomonas, Vibrio spp., acetic acid bacteria, Legionella etc. Other notable groups of Gram-negative bacteria include the cyanobacteria, spirochetes, green sulfur, and green non-sulfur bacteria.

Medically relevant Gram-negative cocci include the four types that cause a sexually transmitted disease (Neisseria gonorrhoeae), a meningitis (Neisseria meningitidis), and respiratory and community-acquired respiratory infections (Moraxella catarrhalis, Haemophilus influenzae).

Medically relevant Gram-negative bacilli include a multitude of species. Some of them cause primarily respiratory problems (Klebsiella pneumoniae, Legionella pneumophila, Pseudomonas aeruginosa), primarily urinary problems (Escherichia co/i, Proteus mirabilis, Enterobacter cloacae, Serratia marcescens), and primarily gastrointestinal problems (Helicobacter pylori, Salmonella enteritidis, Salmonella typhi).

Gram-negative bacteria associated with hospital-acquired infections include Acinetobacter baumannii, which cause bacteremia, secondary meningitis, and ventilator-associated pneumonia in hospital intensive-care units. In addition, Pseudomonas aeruginosa, Klebsiella pneumoniae and Enterobacteraciae are predominant hospital-acquired agents of ventilator-associated pneumonia, along with Acinetobacter.

Various Gram-positive bacteria meet this criteria and may also be used in the present methods. A representative example of such Gram-positive bacterium is Staphylococcus aureus.

A list of pathogens with T3SSs and their host effects follows (reproduced from Coburn et al. 2007. Clin. Microbiol. Rev. 20: 535-549):

| | T3SS components | | | | |
|---|---|---|---|---|---|
| | Structural proteins/ | | | Relationship | Diseases caused |
| Pathogen | translocators | Effectors | Hosts | with hosts | by agent |
| Yersinia species | Ysc injectisome, | YopH, -E, -T, | Humans, | Pathogen | Plague (bubonic, |
| (Y. pestis, Y. | YopB, YopD, LcrV | and -O and | cattle, | | pneumonic, and |
| enterocolitica, Y. | | YpkA, -P/J, | rodents, fleas | | septicemic) (Y. |
| pseudotuberculosis) | | and -M | (Y. pestis) | | pestis), |
| | | | | | enterocolitis and |
| | | | | | mesenteric |
| | | | | | lymphadenitis (Y. |
| | | | | | enterocolitica and |
| | | | | | Y. pseudotuberculosis) |
| Salmonella | SPI1, PrgK, PrgH, | SPI1, AvrA, | Humans, | Pathogen (in | Enterocolitis in |
| entericaserovars | InvG (ring base), | SipA/B/C/D, | rodents, | humans, | humans and |
| (Typhimurium, | PrgI (needle), and | SlrP, SseK, | chickens, | rodents, cows, | typhlitis and |
| Typhi, Paratyphi, | SipB/C/D (putative | SopA/B/D/E/ | cows, and | and pigs), | typhoid-like |
| Sendai, Dublin, | translocators); SPI2, | E, and SptP; | pigs | innocuous | disease in mice |
| and Choleraesuis) | Ssa proteins | SPI2, SpiC, | | carriage (in | (serovar |
| | (apparatus), Ssc | SseF/G/I/J, | | chickens and | Typhimurium), |
| | proteins (effector | SlrP, | | some human | enteric fever in |
| | chaperones), SsrAB | SspH1/H2, | | cases) | humans (serovars |
| | (regulators), and | SifA, SifB, | | | Typhi, Paratyphi, |
| | SseB/C/D | PipB/B2, | | | and Sendai), |
| | (translocators) | SseK1/K2, | | | intestinal |
| | | GogB, and | | | inflammation and |
| | | SopD2 | | | bacteremia in cows |
| | | | | | (serovar Dublin), |
| | | | | | septicemia in pigs |
| | | | | | (serovar |
| | | | | | Choleraesuis) |
| EPEC/EHEC | EspA/B/D | Tir, Map, | Humans, | Pathogen (in | Intestinal |
| | (translocators) | Nle's, | cows, calves | humans and | inflammation and |
| | | EspF/G, Cif, | | calves), | bloody diarrhea |
| | | Orf3 | | innocuous | (EPEC/EHEC), |
| | | | | carriage (in | possibility of renal |
| | | | | cows) | failure and septic |
| | | | | | shock (EHEC) |
| Shigella species | Mxi/Spa (apparatus), | IpaA/B, C | Humans | Pathogen | Bacillary dysentery |
| (S. dysenteriae, S. | IpaB/C | terminus of | (only known | | (shigellosis), |
| flexneri, S. boydii, | (translocators), IpgC | IpaC, VirA, | reservoir) | | sporadic dysentery |
| and S. | (IpaB/C chaperone) | IpaH, Osp's, | | | pandemics (S. |
| sonnei[multiple | | IpgB1 | | | dysenteriae) |
| serotypes]) | | | | | |
| Bordetella species | BopB, BopD | BopC | Humans, | Pathogen | Whooping cough |
| (B. pertussis, B. | (potential | | dogs, pigs | | (B. pertussis and B. |
| parapertussis, | translocators) | | | | parapertussis [milder |
| and B. | | | | | with B. |
| bronchiseptica)^{a} | | | | | parapertussis]), |
| | | | | | kennel cough in |
| | | | | | dogs, atrophic |
| | | | | | rhinitis in swine, |
| | | | | | possible respiratory |
| | | | | | illness in humans |
| | | | | | (B. bronchiseptica) |
| Pseudomonas | PopB and PopD | ExoS, ExoT, | Part of | Opportunistic | Pneumonia |
| aeruginosa | (translocators), PcrV, | ExoU, ExoY | normal flora | and nosocomial | (common cause of |
| | SpcU (chaperon for | | in up to 20% | pathogen | hospital-acquired |
| | ExoU) | | of humans, | | pneumonia and |
| | | | common in | | occasionally of |
| | | | the | | community- |
| | | | environment | | acquired |
| | | | | | pneumonia), |
| | | | | | chronic airway |
| | | | | | infection in cystic |
| | | | | | fibrosis, urinary |
| | | | | | tract infections in |
| | | | | | long-term care |
| | | | | | facilities, various |
| | | | | | other clinical |
| | | | | | infections (e.g., |
| | | | | | endocarditis) in |
| | | | | | immunocompromised |
| | | | | | patients |
| Burkholderia | T3SS-1, T3SS-2, | BopAB | Environmental | Human | Melioidosis, |
| pseudomallei | T3SS-3 (Bsa) | (putative), | isolate, | pathogen | community- |
| | | BopE (T3SS- | humans | | acquired |
| | | 3) | | | bacteremias and |
| | | | | | pneumonias |
| Vibrio | T3SS1 (V. | VP1680 | Aquatic | Human | Noninflammatory |
| parahaemolyticus, | parahaemolyticus), | (T3SS1), | isolate, | pathogens | secretory diarrhea |
| V. cholerae | T3SS2 (V. | VopA | humans | | (V. cholerae), |
| | parahaemolyticusand | (T3SS2) | | | inflammatory |
| | V. cholerae) | | | | diarrhea with |
| | | | | | potential systemic |
| | | | | | spread (V. |
| | | | | | parahaemolyticus) |
| Chlamydia species | YscN (ATPase), | IncA and | Obligate | Human | Sexually |
| | LcrH1 and -2 and | additional Inc | intracellular | pathogens (C. | transmitted |
| | SycE (chaperones), | proteins, | pathogens. | trachomatisand | infection (C. |
| | LcrE (structural | Cpn0909, | infectious | C. pneumoniae), | trachomatis), |
| | “lid”) | Cpn1020 | bodies found | bird pathogen | pneumonia (C. |
| | | | in the | (C. psittaci) | pneumoniae), |
| | | | environment | | psittacosis in birds |
| | | | | | (C. psittaci) |

### T3SS and Amyloid Effects

Pseudomonas aeruginosa expresses a T3SS that injects cytotoxic exoenzyme effectors into host cells. T3SS effectors exoenzymes S, T, U, and Y have been previously described. Exoenzymes S and T are not frequently associated with virulent strains. Exoenzyme U (ExoU), a phospholipase A2, is indicative of highly virulent strains but is only found in roughly 18% of nosocomial strains. However, exoenzyme Y (ExoY) is an adenylyl cyclase with both purine and pyrimidine activity that is found in approximately 90% of nosocomial Pseudomonas strains. Further, ExoY intoxication of pulmonary endothelial cells mediates tau hyperphosphorylation and microtubule breakdown while both ExoU and ExoY promote the formation and extracellular release of cytotoxic tau amyloid oligomers. Of significant importance in the instant disclosure, ExoU activity decreases the innate immune amyloid response whereas ExoY intoxication effectively abolishes the antimicrobicity of endothelial amyloids. Following the suppression of endothelial amyloid antimicrobial activity, ExoY and ExoU intoxication instigates the formation of cytotoxic amyloid prions that may contribute to end-organ dysfunction, neurocognitive decline, and increased morbidity and mortality of critically ill patients recovering from nosocomial pneumonia post-discharge. (Thus, T3SS effector intoxication drives the conversion of antimicrobial endothelial amyloids into cytotoxic amyloid prions. Amyloids comprise the majority of prions.) The neutralization or depletion of oligomeric tau, as disclosed herein, effectively rescues the antimicrobial phenotype of endothelial amyloids and concomitantly abolishes the cytotoxic amyloid prions that arise secondary to the majority of P. aeruginosa mediated nosocomial pneumonia. In addition, tau oligomer neutralization or depletion appears to augment the efficacy of endothelial amyloid antimicrobial activity.

### Host-Pathogen Interactions

The type 3 secretion system and its effectors are well recognized virulence determinants. In Applicants' screen of patients harboring P. aeruginosa infections in the intensive care unit, 106 bacterial strains encoded for a functional type 3 secretion system; only 3 strains did not utilize this virulence mechanism (data not shown). However, how these exoenzymes acquired a functional tertiary structure and then elicited signals to change cell behavior have remained poorly understood. In the instant disclosure, a novel mechanism of host-pathogen interaction has been discovered and described, and with such discovery, a previously unknown role for type 3 secretion system effectors has been identified. As shown herein, infection of cells from the alveolar-capillary membrane elicited production of amyloid proteins. Type 3 secretion system effectors, including most prominently exoenzymes U and Y, change the nature of the amyloid from one that has antimicrobial properties to one that has cytotoxic effects.

Pseudomonas aeruginosa is a common cause of pneumonia that progresses to sepsis and acute respiratory distress syndrome. Pseudomonas displays a vascular tropism with hemorrhagic lesions in the pulmonary microcirculation. This histopathological pattern is described as a vasculitis and coagulative necrosis. Much of the acute virulence of this organism has been largely attributed to the presence of a type 3 secretion system and its effector exoenzymes (Pseudomonas aeruginosa injects cytotoxic effectors directly into host cells via a T3SS—see, e.g., Izore et al., 2011. Structure, 19: 603-12). The P. aeruginosa exoenzymes include ExoS, ExoT, ExoU, and ExoY. Because P. aeruginosa injects these exoenzymes into lung alveolar epithelial cells and microvascular endothelial cells during infection, exoenzymes have been utilized herein to reveal the fundamental nature of these cell types, especially the organization of signal transduction networks, to better resolve mechanisms of endothelial innate immunity, and to dissect basic principles of endothelial cell heterogeneity.

Among the exoenzymes, ExoU is generally believed to endow the greatest virulence. ExoU is a phospholipase A2 enzyme, and so it breaks down cell membranes leading to cell lysis. ExoU also generates cytotoxic amyloids. ExoY causes cell rounding that, by comparison with ExoU, does not reflect the extreme degree of cytotoxicity. However, ExoY generates the most cytotoxic amyloid admixture. Therefore, ExoY is an important virulence determinant, and its activity may be most relevant to cellular injury and repair in the aftermath of infection. The importance of ExoY to infection in the clinical setting has been questioned. Prior studies seemed to suggest that the presence of ExoY contributed little to the severity of the initial infection. However, it has now been realized via the current studies that the detrimental impact of ExoY extends beyond this initial phase.

ExoY is the most recently identified type 3 secretion system effector. It was originally described as an adenylyl cyclase, but ExoY is now recognized as a promiscuous purine and pyrimidine nucleotidyl cyclase. Work with this enzyme has revealed the importance of compartmentalized cAMP signaling in endothelium, and has contributed to: (1) the instant understanding of how microtubules regulate endothelial barrier integrity (2) discovery of multiple endothelial cell microtubule associated proteins, including an endothelial tau, (3) discovery that tau and other insoluble proteins are released from pulmonary endothelium during infection to cause a transmissible proteinopathy; and (4) that endothelium produces pyrimidine cyclic nucleotides. Thus, ExoU and ExoY signaling mechanisms are now being evaluated in mammalian cells, specifically seeking the impact of such signaling upon infection in vitro, in all organ systems, and in animals and humans.

### Microbial Prevalence of Cytotoxic Amyloid Oligomer Induction

As disclosed herein, results for Staphylococcus aureus and Klebsiella pneumoniae indicate that the induced release of cytotoxic endothelial amyloid oligomers is common to nosocomial pathogens. These pathogenically elicited cytotoxic amyloid oligomers suppress or abolish the antimicrobial amyloid response component of the innate immune system (emerging evidence has indicated that amyloids function in innate immunity). This exacerbates nosocomial virulence and constitutes a pulmonogenic amyloid prion disease. Taken together, these mechanisms promote morbidity and mortality prior to discharge and likely contribute to neurocognitive decline, neurodegeneration, and end-organ dysfunction common in ICU patients recovering from nosocomial pneumonia post-discharge. The ability to reverse the highly pathogenic amyloid prion phenotype and rescue antimicrobial activity post-infection indicates significant therapeutic promise especially in light of increasing trends of antibiotic resistance. Explicitly contemplated therapeutic applications of the instant disclosure include prophylactic or post-infection treatment of nosocomial pneumonia, sepsis, superficial infections, and/or burns. Further, amyloid immunodepletion, as has been experimentally examined herein, can readily lend itself to increasing the efficacy of antimicrobial peptide treatments currently in use.

### Prions

Prions are proteins that acquire alternative conformations that become self-propagating. Generally, such conformational changes are characterized by increased β-sheet structure and a propensity to aggregate into oligomers (Prusiner. 2013. Annu. Rev. Genetics, 47: 601-23). Prions are, by definition, transmissible and self-replicating.

### Pulmonary Endothelial Amyloids

Pulmonary endothelial amyloids, as disclosed herein, exhibit broad-spectrum antimicrobial activity following bacterial challenge. These amyloid species are effectors of the endothelial innate immune response and induce significant bacteriostasis against prevalent nosocomial pathogens Pseudomonas aeruginosa, Klebsiella pneumoniae, and Staphylococcus aureus, and possibly many others—indeed all other microbes are under consideration for treatment with the amyloid assemblies of the current disclosure, in view of evidence obtained. In particular, pulmonary endothelial amyloids have also been identified herein as effective against the predominant nosocomial fungal pathogen Candida albicans as well as E. coli, MRSA, and Klebsiella pneumoniae. The assemblies of the instant disclosure are being tested against a wide variety of nosocomial pathogens (e.g., Acinetobacter) as well as the influenza virus. Current evidence suggests that the efficacy of the amyloid assemblies of the instant disclosure against both Gram-positive and Gram-negative bacteria will differ only in the extent of the static/cidal effect and the time required to exert that effect. The production and enrichment of antimicrobial endothelial amyloids is therefore a promising novel therapeutic option for antibiotic resistant organisms and nosocomial pneumonia with the potential to abrogate increased rates of end-organ dysfunction, neurocognitive decline, and early mortality seen among critically ill patients post-discharge. However, P. aeruginosa T3SS exoenzyme intoxication of host cells abolishes the antimicrobicity of endothelial amyloids. In the absence of the injection of T3SS-mediated exoenzymes into the host cell, as disclosed herein, P. aeruginosa infection of endothelial cells is sufficient to induce the release of potent antimicrobial endothelial amyloid species. Antimicrobial endothelial amyloids have been generated herein via the infection of endothelial cells with a strain of P. aeruginosa that has a non-functional T3SS which lacks the ability to inject T3SS-mediated exoenzymes into the host cell. Following a period of incubation, the supernatant from the infected cells has been collected, centrifuged, and filter-sterilized. This method of the instant disclosure readily lends itself to large-scale production via currently existing industrial cell culture technologies. When derived from the infection of human endothelial cells, antimicrobial amyloids are likely to possess a large margin of safety with negligible risk of eliciting an immunogenic response in patients. Additional contemplated applications for antimicrobial endothelial amyloids of the instant disclosure produced in this manner include the treatment of burns, wounds, sepsis, cystic fibrosis, and infection.

The method of endothelial amyloid production of the instant disclosure is straightforward and can be readily adapted to large scale production. With human endothelial cells employed, the potential for the treatment to elicit an immunogenic response in patients can be significantly minimized. This method can also be adapted via different cell types for supernatant substrate to produce treatments for veterinary use as well as human use.

It is noted that WO 2010/105191 (PCT/US2010/027186) identified synthetic Aβ peptides as possessing anti-microbial activity. However, the instant preparations can be readily distinguished from such synthetic Aβ peptides in both their complexity (with certain compositions of the instant invention providing a naturally-produced, complex assemblage of amyloid and accompanying peptides/factors) and in their efficacy (e.g., the instant compositions have exhibited biofilm-directed antimicrobial activity, which has not been described for isolated/synthetic Aβ peptides).

### Cell Culture and Passage

Mammalian cell culture can be performed by methods known in the art. Certain aspects of the instant disclosure employ mammalian endothelial cells and HBSS cell culture media. A range of other cell types and cell culture media can also be used/substituted for performance of the cell culture and passage events disclosed herein, such as for example, mammalian arterial endothelial cells. For example, the amyloid complexes of the instant disclosure can also be generated through cells cultured in DMEM. It is likely that all mammalian cells can produce the amyloid assemblies of the instant disclosure; however, efficacy is likely to be highly variable, based upon the specific cell's expressed proteins. For purposes of sequential depleting or neutralizing of tau and Aβ amyloid species, antibodies known in the art that specifically bind these peptide species may be advantageously used. Representative examples of such antibodies, commercial sources, and representative amounts for use, are set forth in the following table:

| Antibody | Company/Lab | Catalog # | Dilution | References |
|---|---|---|---|---|
| Pan-Tau (Tau-5) | MBL | AT-5004 | 1:500- | Castillo-Carranza et al., |
| monoclonal | | | 1:2500 | J. Neurosci., 2012 |
| pS214-Tau | MBL | AT-5018 | 1:500- | Jamsa et al., |
| polyclonal | | | 1:2500 | Biochem. Biophys. Res. |
| | | | | Comm., 2004 |
| Tau R1 | Lester Binder | R1 | 1:500- | Kanaan et al., J. |
| monoclonal | Nick Kanaan | | 1:2500 | Neuropathol. Exp. |
| | Lab | | | Neurol., 2016 |
| TOC-1 | Lester Binder | TOC1 | 1:500- | Ward et al., J. |
| monoclonal | Nick Kanaan | | 1:2500 | Alzheimers Dis., 2013 |
| | Lab | | | |
| TNT-1 | Millipore-Sigma | MABN471 | 1:500- | Combs et al., Neurobiol |
| monoclonal | | | 1:2500 | Dis., 2016 |
| TNT-2 | Millipore-Sigma | MABN2406 | 1:500- | Combs et al., Neurobiol |
| monoclonal | | | 1:2500 | Dis., 2016 |
| T22 | Millipore-Sigma | ABN454 | 1:500- | Lasagna-Reeves et al., |
| polyclonal | | | 1:2000 | FASEB J., 2012 |
| A11 | Stressmarq | SPC-506D | 1:100- | Morgado et al., PNAS, |
| polyclonal | | | 1:1500 | 2012 |
| OC | Millipore-Sigma | AB2286 | 1:500- | Kayed et al., Mol. |
| polyclonal | | | 1:5000 | Neurodegener., 2007 |
| β-amyloid 1-43 | Novus | NBP2-25093 | 1:500- | Stieren et al., J. Biol. |
| polyclonal | | | 1:2500 | Chem., 2011 |
| MOAB -2 | Novus | NBP2-13075 | 1:500- | Youmans et al., Mol. |
| monoclonal | | | 1:5000 | Neurodegener., 2012 |
| β-amyloid 1-40 | Biolegend | 805401 | 1:500- | Portelius et al., J. |
| monoclonal | | | 1:2500 | Proteome Res., 2006 |
| β-amyloid 1-42 (1F4) | Bioss Antibodies | bsm-0107M | 1:500- | Hrncic et al., Am. J. |
| monoclonal | | | 1:2500 | Pathol., 2000 |

### Filter Sterilization

In certain aspects of the instant disclosure, primary culture harvests are filter-sterilized prior to further use/propagation. Exemplary pore sizes that can be used for filter-sterilization of, e.g., harvested culture media include 0.22μ, 0.2μ, 0.1μ, or less.

### Personalized Therapies

In certain aspects of the instant disclosure, cells (e.g., endothelial cells) can be harvested from a subject having or at risk of developing a microbial infection, sepsis, burn(s), etc., and the harvested cells can then be used to produce antimicrobial amyloid preparations, for subsequent personalized treatment of the subject (with such “self” cell preparations presenting little or no risk of inducing negative immune-related effects). In such aspects, cells harvested from a subject, e.g., can be infected with Pseudomonas aeruginosa that possess a T3SS of reduced, compromised or no activity, thereby promoting production and release of antimicrobial amyloids by the harvested cells of the subject. Antimicrobial amyloid-containing supernatants of such cells can then be collected, and optionally passaged further, as is described elsewhere in the instant disclosure. These antimicrobial amyloid-containing preparations can then be administered using methods known in the art and/or described elsewhere herein.

In certain embodiments, it is expressly contemplated that ex vivo amyloids can be generated ahead of time, banked (i.e., frozen, as the efficacy of these amyloids is not reduced by freeze thaw cycles), or stored so that they can be available in case that individual ever became critically ill or in need of them. This can be done for little cost. Such therapies are projected to be invaluable for individuals that contract HIV (HIV patients are highly immunocompromised and usually die from repeated infections), suffer serious burns, are hospitalized for critical injuries (e.g., due to an MVA) and contract nosocomial pneumonia, elderly patients, etc.

Monoclonal anti-tau antibodies specific to the oligomeric conformation can also be used as an infusion for critically ill patients who contract nosocomial pneumonias to capture and neutralize tau oligomers. In certain embodiments, such an approach is provided to augment the ability of the patient to fight infection (it would remove the inhibitory tau that nullifies the antimicrobial activity of the patient's own endothelial amyloid complexes during infection). Further, it is likely to reduce the neurocognitive decline and secondary end-organ damage common in survivors of critical illness and nosocomial infection.

In certain embodiments, for delivery of such agents, it is contemplated that the agents of the instant disclosure are immunopurified, concentrated and solubilized in salt solution. Concentration is calculated in Units (much like Thrombin, for example). The agents can also be incorporated into a paste for topical application, much like steroid cream and/or incorporated into implants, such as stents, e.g., for vascular indications, or into plastic and metal, e.g., for orthopedic consideration.

### Pharmaceutical Compositions

Agents and/or cell-culture-derived preparations (e.g., antimicrobial amyloid preparations) of the present disclosure can be incorporated into a variety of formulations for therapeutic use (e.g., by administration) or in the manufacture of a medicament (e.g., for treating or preventing a microbial infection, sepsis, burn(s), etc.) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, such as an antimicrobial amyloid preparation, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent or preparation described herein may be used (e.g., administered to an individual, such as a human individual, in need of treatment with an amyloid antimicrobial preparation) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure (amyloid complex-containing preparations and/or optionally antimicrobial amyloid complexes isolated from cell culture supernatants), normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 ag/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such as magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., an antimicrobial amyloid complex and/or preparation) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively, or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively, or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Drugs provided herein can be formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for oral delivery or intravenous injection to a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., an antimicrobial amyloid complex/assembly and/or preparation) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

A drug or other therapy of the instant disclosure is administered to the subject in an amount sufficient to achieve a desired effect at a desired site (e.g., reduction of microbial abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., an antimicrobial amyloid complex/assembly and/or preparation) described herein. In certain embodiments, a dose described herein includes independently between 1 mg and 3 mg, inclusive, of an agent (e.g., an antimicrobial amyloid complex/assembly and/or preparation) described herein. In certain embodiments, a dose described herein includes independently between 3 mg and 10 mg, inclusive, of an agent (e.g., an antimicrobial amyloid complex/assembly and/or preparation) described herein. In certain embodiments, a dose described herein includes independently between 10 mg and 30 mg, inclusive, of an agent (e.g., an antimicrobial amyloid complex/assembly and/or preparation) described herein. In certain embodiments, a dose described herein includes independently between 30 mg and 100 mg, inclusive, of an agent (e.g., an antimicrobial amyloid complex/assembly and/or preparation) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg. It will be also appreciated that an agent (e.g., an antimicrobial amyloid complex/assembly and/or preparation) or composition, as described herein, can be administered in combination with one or more additional pharmaceutical agents (e.g., therapeutically and/or prophylactically active agents), which are different from the agent or composition and may be useful as, e.g., combination therapies.

The agents or compositions can be administered in combination with additional pharmaceutical agents that improve their activity (e.g., activity (e.g., potency and/or efficacy) in treating a disease (e.g., a microbial infection) in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent (e.g., an antimicrobial amyloid complex/assembly and/or preparation) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of an antimicrobial peptide treatment, other antimicrobial therapy, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, additional antimicrobial agents, immunotherapy and/or immunomodulatory agents, anti-proliferative agents, cytotoxic agents, anti-angiogenesis agents, anti-inflammatory agents, immunosuppressants, anti-viral agents, cardiovascular agents, cholesterol-lowering agents, anti-diabetic agents, anti-allergic agents, contraceptive agents, and pain-relieving agents. In certain embodiments, the additional pharmaceutical agent is an antimicrobial agent. In certain embodiments, the additional pharmaceutical agent is an antibiotic. It is further contemplated that in the Cystic Fibrosis lung, the amyloid assemblies of the instant disclosure can be used in combination, e.g., with DNase and/or other CF-directed therapies (e.g., inhaled saline, etc.). In certain embodiments, the agents described herein or pharmaceutical compositions can be administered in combination with another antimicrobial therapy, e.g., an antimicrobial peptide treatment and/or other antimicrobial therapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Combination Treatments

The compositions and methods of the present disclosure may be used in the context of a number of therapeutic or prophylactic applications. In order to increase the effectiveness of a treatment with the compositions of the present disclosure, e.g., an antimicrobial preparation selected and/or administered as a single agent or preparation, or to augment the efficacy of another therapy (second therapy), it may be desirable to combine these compositions and methods with one another, or with other agents and methods effective in the treatment, amelioration, or prevention of diseases and pathologic conditions, for example, nosocomial infection, sepsis, superficial infection(s), burn(s), etc.

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

### Kits

The instant disclosure also provides kits containing agents of this disclosure for use in the methods of the present disclosure. Kits of the instant disclosure may include one or more containers comprising an agent (e.g., an antimicrobial amyloid complex/assembly and/or preparation) of this disclosure and/or may contain agents (e.g., test plates, oligonucleotide primers, probes, etc.) for identifying a drug-resistant microbial infection for which application of an agent of the instant disclosure would be advantageous. In some embodiments, the kits further include instructions for use in accordance with the methods of this disclosure. In some embodiments, these instructions comprise a description of administration of the agent to treat or diagnose, e.g., a drug-resistant microbial infection, according to any of the methods of this disclosure. In some embodiments, the instructions comprise a description of how to detect a drug-resistant microbial infection for treatment, for example in an individual, in a tissue sample, or in a cell. The kit may further comprise a description of selecting an individual suitable for treatment based on identifying whether that subject has a drug-resistant microbial infection.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a drug-resistant microbial infection, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, at least one active agent in the composition is an antimicrobial amyloid complex/assembly and/or preparation. The container may further comprise a second pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Generation of Primary Antimicrobial Supernatant

PMVECs were grown to confluence in Dulbecco's Modified Eagle Medium (DMEM) supplemented with 10% fetal bovine serum and 5% penicillin/streptomycin and incubated at 37° C. in 5% carbon dioxide. To prepare the bacterial inoculant, T3 SS-deficient P. aeruginosa was streaked onto Vogel-Bonner minimal salts media agar (without glucose) from frozen stocks and grown overnight at 37° C. prior to the day of infection. The day of infection, the media was removed from confluent monolayers which were then rinsed with Hank's Balanced Salt Solution (HBSS) and incubated during the preparation of the bacteria. T3SS-incompetent P. aeruginosa was obtained from the overnight plate and suspended in 1× phosphate buffered saline (pH 7.4) to an OD₅₄₀ of 0.25 (previously determined to be 2E8 CFUs/mL). The bacterial suspension was then diluted in HBSS to achieve a multiplicity of infection of 20:1. Next, the HBSS was removed from the incubated monolayers which were then treated with the 20:1 bacterial suspension and incubated for 4 h post-infection. Supernatants were then collected, centrifuged at 4500 rpm, and subsequently sterilized via passage through a 0.22 μm PES filter.

### Passaging of Antimicrobial Amyloids

Confluent PMVEC or PAEC monolayers were rinsed with HBSS and treated with bacteria-free primary antimicrobial supernatant (derived from infection of PMVECs or PAECs with T3SS-deficient P. aeruginosa). Treated monolayers were then incubated for 4 h. The primary supernatant was then removed and the monolayer rinsed 5× with HBSS. After rinsing, the monolayer was treated with a fresh layer of media (50% clear DMEM) and incubated for 20 h. The supernatant was then collected, centrifuged at 4500 rpm, and filter-sterilized with a 0.22 μm PES filter. This process may be repeated indefinitely. The enrichment of the antimicrobial amyloid component may be achieved through longer incubation periods.

### Antibody Neutralizations and Immunodepletions

Pre-cleared supernatants were neutralized through the addition of either A11 (Stessmarq), T22 (Millipore), Aβ₁₋₄₀ (Biolegend), or Aβ₁₋₄₃ antibody (Novus) at 1:500 and then rotated overnight at 4° C. Protein agarose beads were then added to each solution at 1:1000 and followed by rotation for an additional 3 h at 4° C. to pull down the antibody captured amyloids. Solutions were then centrifuged at 4500 rpm for 10 minutes and supernatants were then filter-sterilized as previously described. After filtering, neutralized supernatants were dialyzed against 4 changes of HBSS, collected, and sterilized by passage through a 0.22 μm PES membrane syringe filter.

### Isolation of Antibody Captured Amyloids for Add-Back Experiments

Collected antibody-bead complexes from immunodepleted/neutralized supernatants were rinsed 6 times with 1×PBS, rinsed 1 time with 0.5 M NaCl in PBS, rinsed 1 time with 0.5 M NaCl in PBS with 0.1% Triton, 1 time with PBS, and then re-suspended to original volume in 4 M MgCl2 to elute captured amyloid-antibody complexes from the agarose beads. Amyloid-antibody complexes were then heated to 95° C. for 10 minutes to denature the antibody and release the amyloid eluates. Next, the solutions are dialyzed against 6 changes of 1×HBSS. Solutions containing the eluted amyloid were collected, re-suspended to original volume in HBSS, and filter-sterilized as noted above for immunodepletions/neutralizations prior to use.

Treatments presented herein included:
Negative or vehicle control treatment: Hank's Balanced Salt Solution (HBSS)
Active Treatments: ExoU-related treatments included the P. aeruginosa PA103 strain (T3SS intact) and the PA103-derived ΔPcrV (possessing a non-functional T3SS). ExoY-related treatments included the P. aeruginosa PA01 strain (T3SS intact), ExoY⁺ (wt), ExoY^{K81M} (possessing a catalytically inactive ExoY), P. aeruginosa PA01 strain-infected PMVEC supernatants from which non-tau amyloid oligomers were immun-depleted, and P. aeruginosa PA01 strain-infected PMVEC supernatants from which tau amyloid oligomers were immune-depleted.

### Example 2: P. aeruginosa Strains Lacking a Functional T3SS (ΔPcrV) Provoked Release of Non-Cytotoxic and Antimicrobial (Including Anti-Biofilm) Compositions from Infected PMVECs

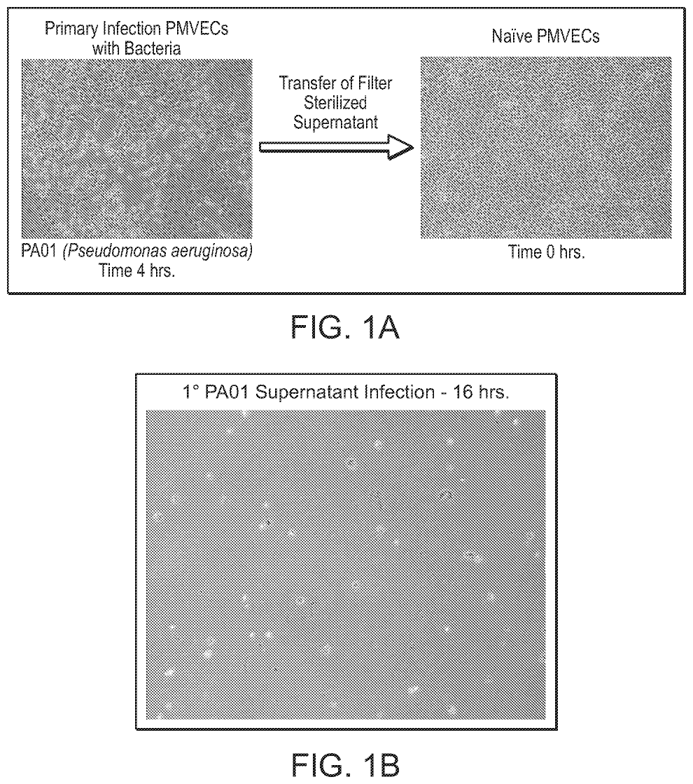

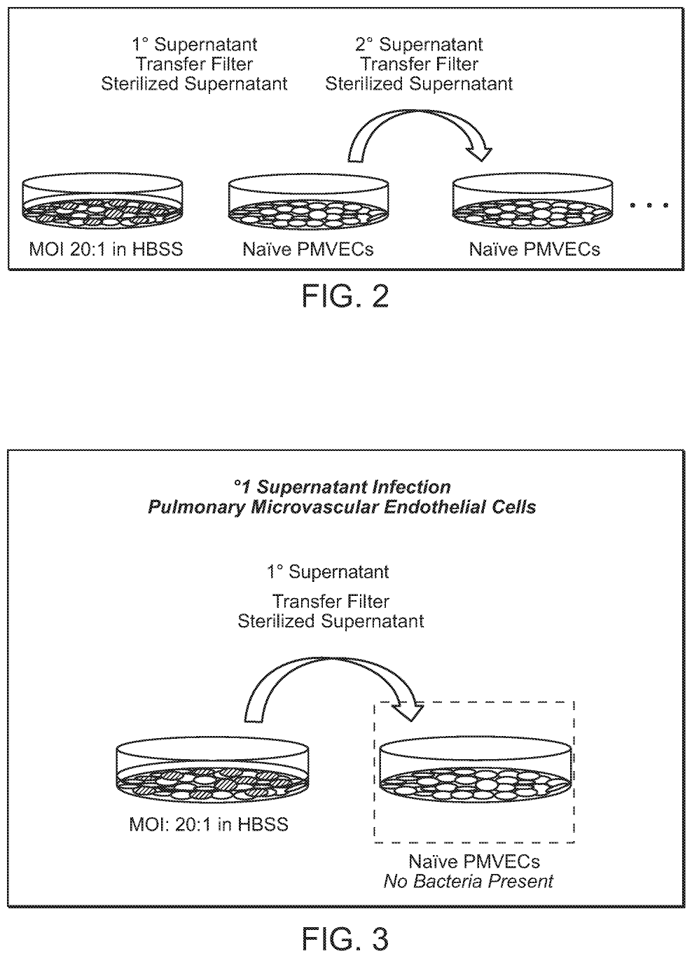

The hypothesis that Pseudomonas aeruginosa T3SS effectors might be sufficient for production of cytotoxic amyloids was initially assessed. To test this hypothesis, P. aeruginosa strains with (PA103 and PA01) and without (ΔPcrV) a functional T3SS were used to infect pulmonary microvascular endothelial cells (PMVECs) for 4 hours at an MOI of 20:1. Supernatants were collected, centrifuged, filter sterilized, and transferred to naïve PMVECs (FIG. 1A). (For certain procedures of the instant disclosure, this initial supernatant was discarded from the P. aeruginosa strain-PMVEC cell admixture at four hours, cells were rinsed four times, and fresh HBSS was provided, with incubation continued for 16-20 hours, thereby producing a secondary supernatant having amyloid assemblies that could then be collected. Optionally, multiple additional passages of supernatant application to naïve cells could be performed (e.g., 2-10 additional passages), in view of the transmissible and self-replicating nature of the observed amyloid assembly effects.) Supernatant obtained from both PA103- and PA01-infected cells was cytotoxic (FIG. 1), though supernatants derived from PA01-infected cells were significantly more cytotoxic than supernatants derived from PA103-infected cells (FIG. 5). As illustrated in FIG. 2, the cytotoxic effect observed for supernatants of PMVECs infected with T3SS P. aeruginosa strain PA01 was self-replicating, and the observed cytotoxic effect was not dependent upon continued bacterial infection, only the presence of supernatant components derived from originally infected PMVECs (FIG. 3, which illustrates a primary supernatant infection performed using PMVECs). Thus, cytotoxic amyloid oligomers induced by P. aeruginosa T3SS effector intoxication were identified as both transmissible and self-replicating. As such, cytotoxic amyloid oligomers were effectively amyloid prions—notably, both amyloid prions and P. aeruginosa-induced cytotoxins were observed to be resistant to RNase, DNase, proteases and heat.

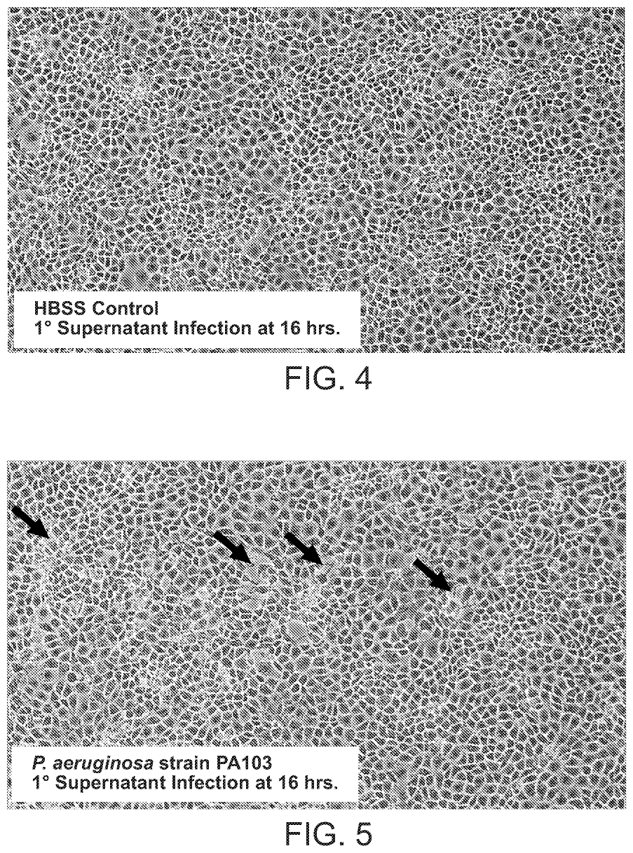

PA103-infected cell supernatants exhibited some cytotoxicity (FIG. 5), as compared to HBSS-treated negative control cells (FIG. 4), even if such cytotoxicity was not as potent as that observed for PA01-infected cell supernatants (see, e.g., FIG. 7).

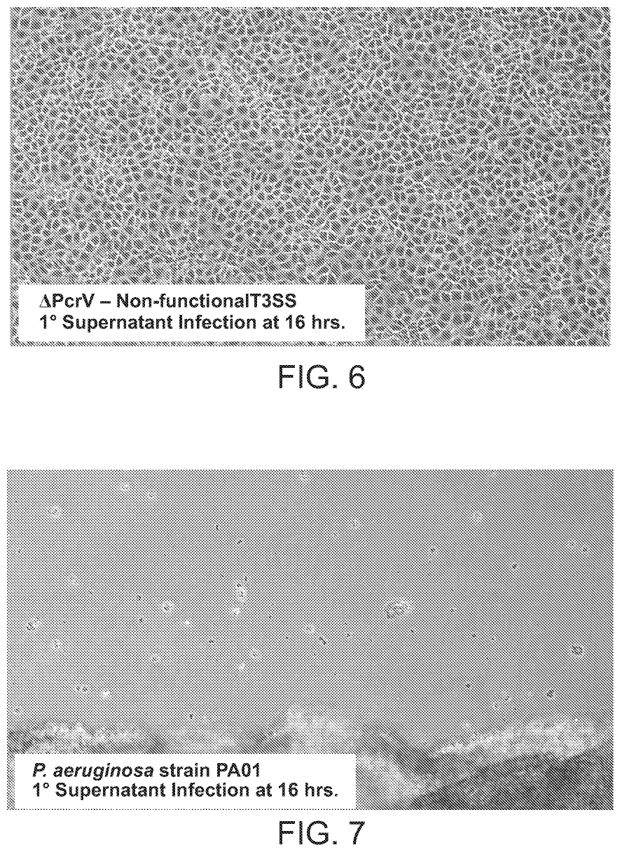

Consistent with T3 SS effectors contributing to cytotoxicity of supernatants produced by cells infected with Pseudomonas aeruginosa possessing active T3SS effectors, a remarkable decline in cytotoxicity was observed for supernatants of cells infected with a Pseudomonas aeruginosa possessing a deletion mutant of T3SS effector (needle tip) protein PcrV (FIG. 6). The PA103 mutant ΔPcrV possesses a non-functional T3SS that prevented injection of effectors into host cells.

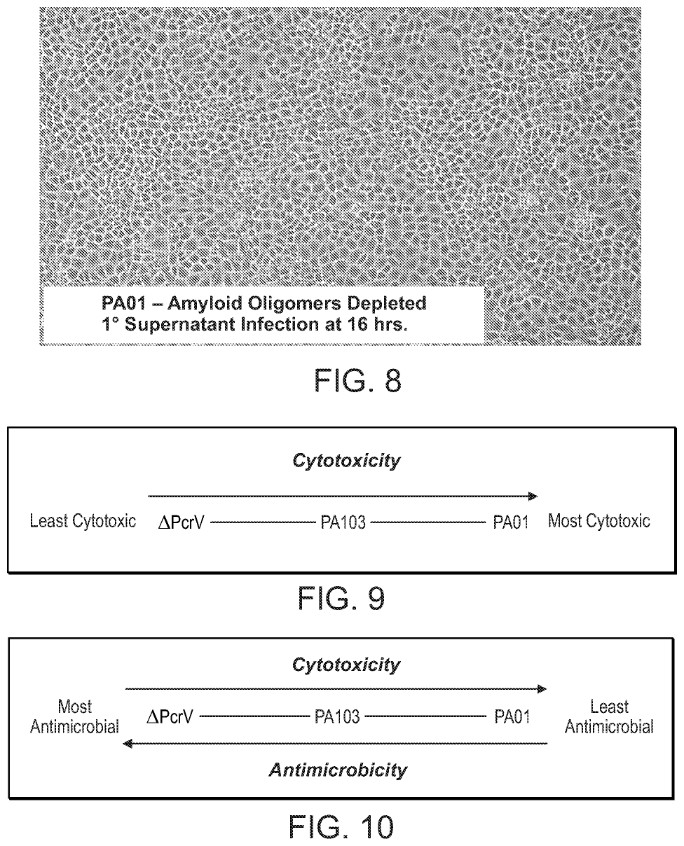

Further supportive of PMVEC-produced amyloids having caused the cytotoxic effects that were observed, amyloid antibody neutralization was conformed as capable of abolishing cytotoxicity (FIG. 8). Meanwhile, cytotoxicity was restored by adding back eluted amyloids (data not shown). In contrast, supernatant obtained from ΔPcrV-infected cells was not cytotoxic (FIG. 6). Consequently, the antimicrobicity of infection-induced endothelial amyloids was examined.

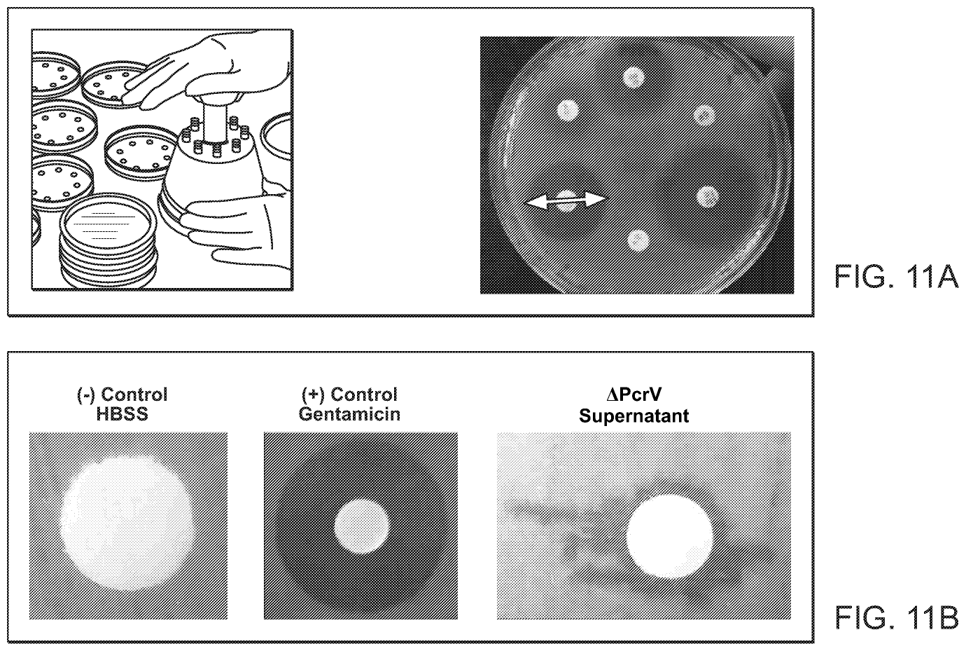

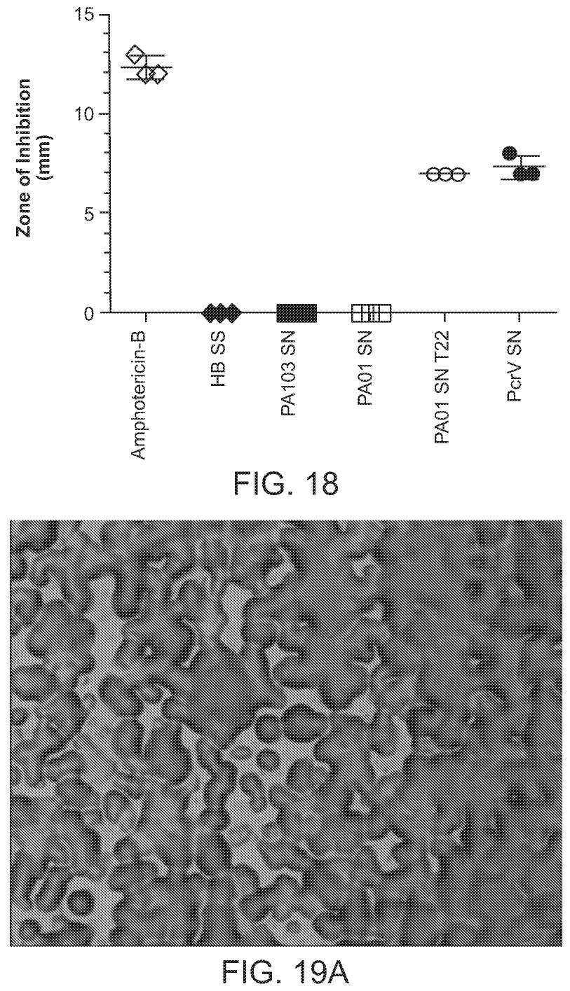

Kirby-Bauer disk diffusion assays (FIG. 11A) were used with disk inoculants standardized to 10 μg/20 μL. Amyloids were visualized by Congo red staining. In contrast to the above cytotoxicity assays, ΔPcrV-derived supernatant effected extensive bacteriostasis, (FIGS. 111B, 12 and 13) whereas wild type PA103- and PA01-derived supernatant had little antimicrobial effect. Bacteriostasis progressively increased over a 72-hour time course (FIG. 13), and was suppressed by amyloid neutralization (FIG. 18, “PA01 SN T22” result). These data indicated that T3SS effectors promoted endothelial amyloid cytotoxicity while, provocatively, suppressing/abolishing antimicrobicity. Antimicrobial compounds, such as those identified herein in ΔPcrV infection-derived supernatants (enriched for antimicrobial amyloids), were therefore identified as a treatment for infections involving antibiotic resistant organisms.

The above-described results identified that both cytotoxicity and antimicrobicity of PMVEC-produced amyloids exist upon separate and inversely related continuums (FIGS. 9 and 10). For antimicrobicity, it was particularly notable that infection of PMVECs by a T3SS-mutated ΔPcrV P. aeruginosa derived from strain PA103 (P. aeruginosa ΔPcrV) induced the release of antimicrobial amyloids from infected PMVECs (FIG. 10).

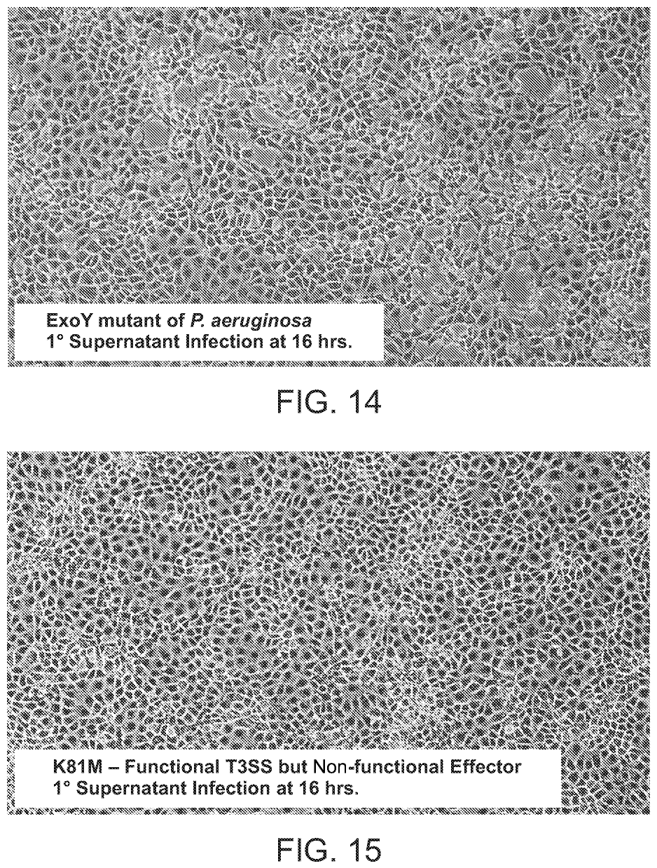

Other mutations of the T3SS/T3SS effector system produced effects upon cytotoxicity and antimicrobicity that paralleled those observed for P. aeruginosa ΔPcrV. PMVEC supernatant obtained from cells infected with an ExoY mutant of P. aeruginosa was observed to possess reduced cytotoxic activity when applied to naïve PMVECs (FIG. 14). Indeed, a particular ExoY mutant, ExoY^{K81M} (possessing a catalytically inactive ExoY, with a functional T3SS but non-functional effector) of P. aeruginosa was observed to have reduced cytotoxic activity when applied to naïve PMVECs (FIG. 15).

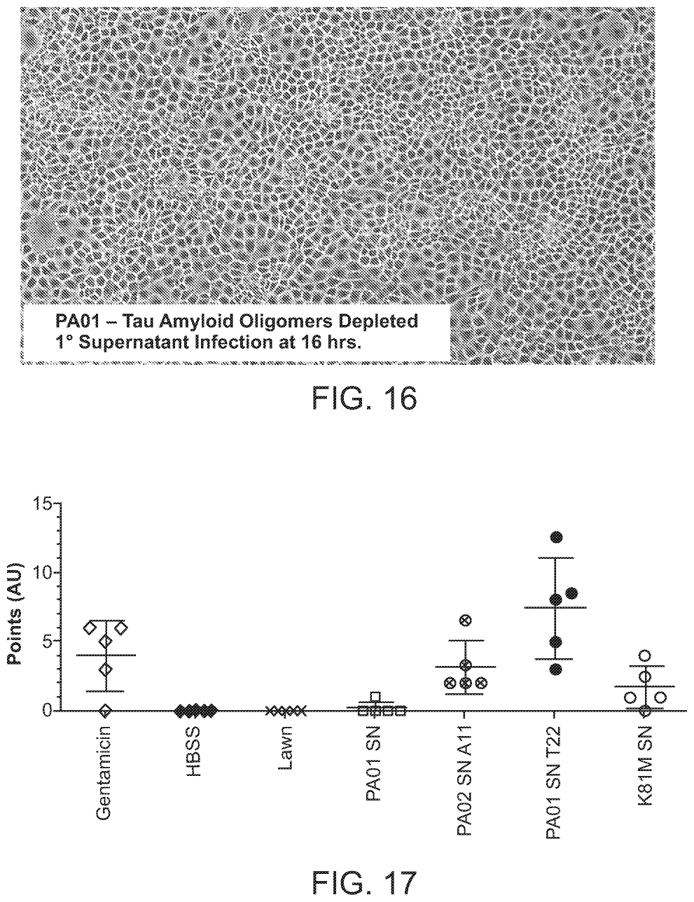

Further to the above-noted observation that amyloid immunodepletion could reduce cytotoxicity of infected PMVEC-derived supernatants, PMVEC supernatant obtained from cells infected with P. aeruginosa strain PA01 which was then immunodepleted for tau amyloid oligomers in particular, exhibited reduced cytotoxic activity when applied to naïve PMVECs (FIG. 16). Immunodepletion of T3SS-induced amyloid oligomers also rescued the antimicrobial activity of endothelial amyloids, post-infection (FIG. 17). Thus, immunodepletion or neutralization of amyloid oligomers was thereby identified as a viable approach for reducing the cytotoxicity of infected PMVEC supernatants, which indicated that clinical application of such anti-amyloid antibodies could also prompt reduced cytotoxicity in infected subjects, thereby providing a therapy for infected subjects otherwise at risk of, e.g., organ damage, etc.

The antimicrobial effects observed for endothelial amyloids derived from endothelial cells (PMVECs) infected with mutant P. aeruginosa strains were further identified to extend to additional classes of microbe. Notably, endothelial amyloids derived from endothelial cells (PMVECs) infected with mutant P. aeruginosa strains PA01 SN T22 and ΔPcrV (PcrV SN) were also demonstrated to inhibit the nosocomial yeast Candida albicans (FIG. 18).

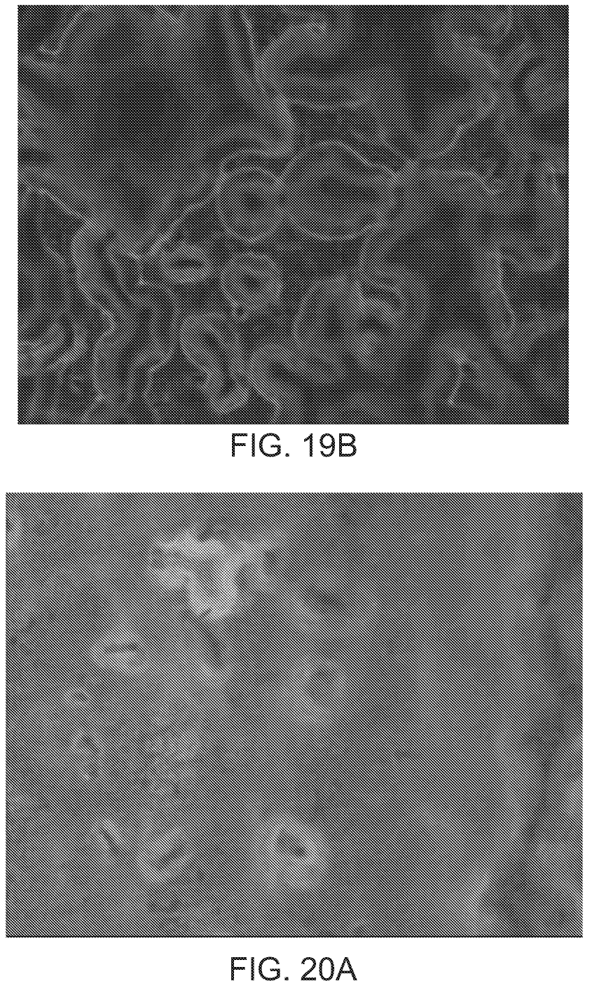

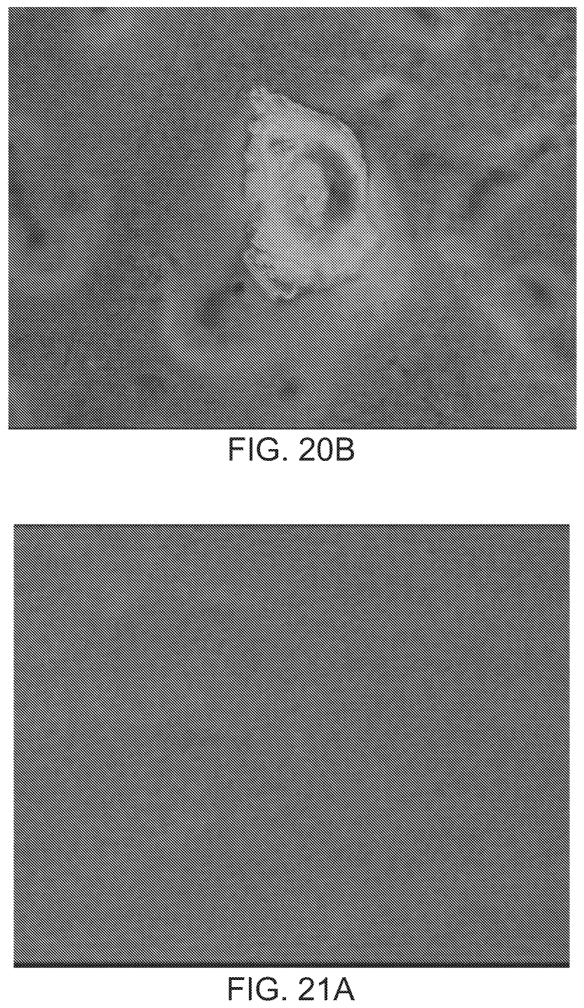

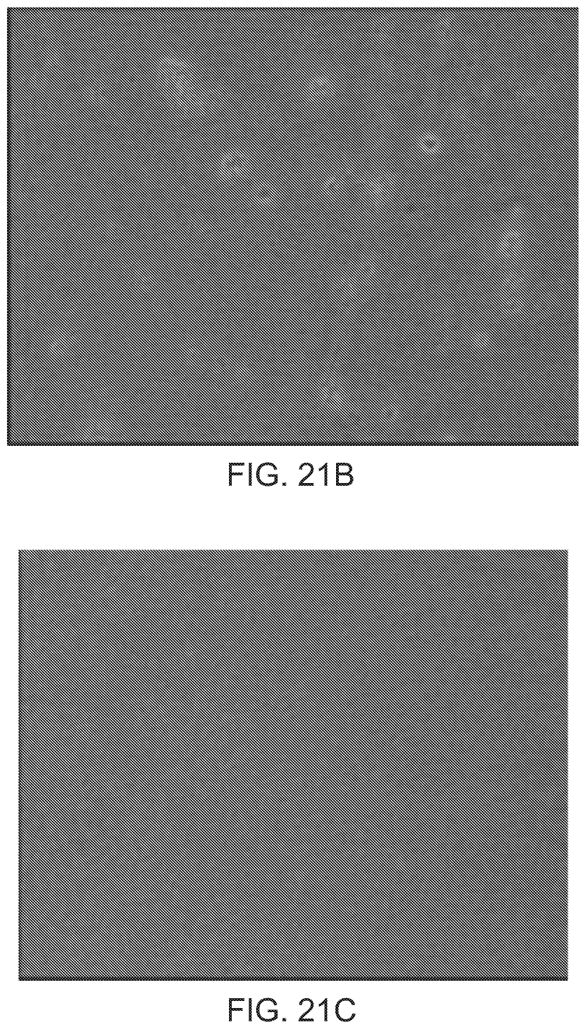

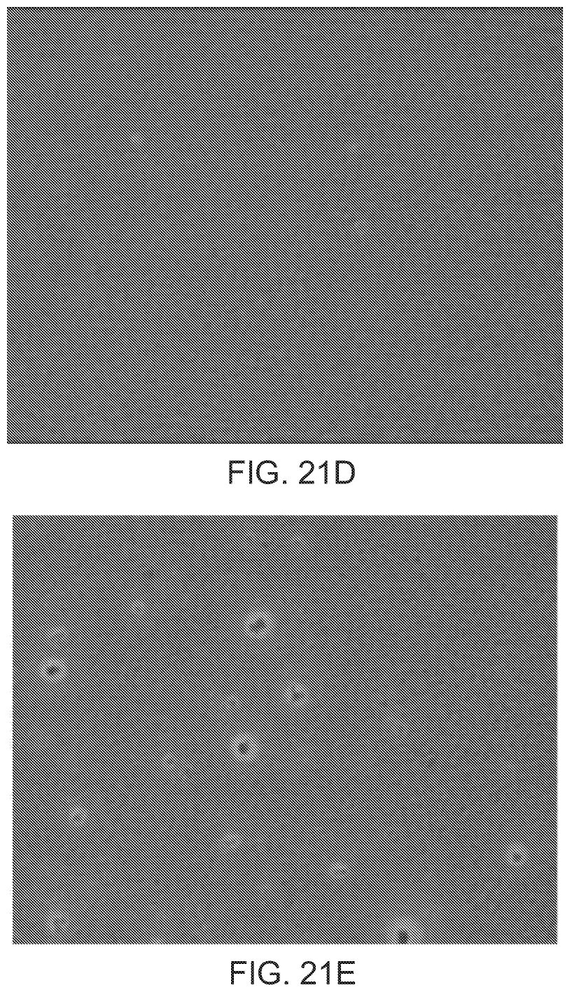

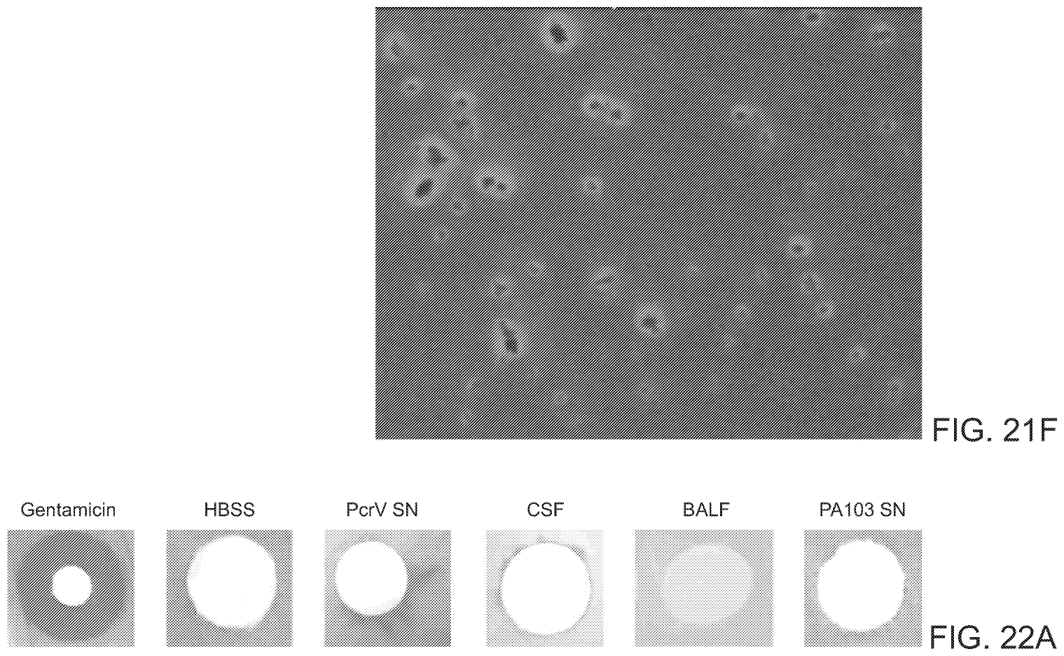

Remarkably, endothelial amyloids were also identified to break down biofilms in a number of strains, particularly P. aeruginosa strain PA01, methicillin-resistant Staphylococcus aureus and Klebsiella pneumoniae (such anti-biofilm effects notably distinguish the amyloid preparations of the instant disclosure from, e.g., antimicrobial effects previously observed for synthetic Aβ). The amyloid-rich biofilm of the P. aeruginosa strain PA01 was broken down via administration of endothelial amyloids (FIGS. 20A and 20B, as compared to FIGS. 19A and 19B). Amyloid immunodepletion (whether tau amyloid directed immunodepletion or non-tau amyloid directed immunodepletion) of otherwise cytotoxic, reduced antimicrobicity supernatants was also observed to rescue anti-biofilm activity of such supernatants (consistent with the antimicrobicity rescue of such immunodepletion treatments observed above) (FIGS. 21E and 21F, as compared to FIG. 21A (negative control), FIG. 21B (positive control, gentamicin), FIG. 21C (HBSS negative control) and FIG. 21D (non-immunodepleted ExoY⁺ supernatant)). While both tau amyloid directed immunodepletion and non-tau amyloid directed immunodepletion produced anti-biofilm efficacy in treated supernatants, tau amyloid directed immunodepletion was notably most effective in these assessments (FIG. 21F).

Anti-biofilm efficacy of infected PMVEC-derived antibodies was further observed not only for P. aeruginosa but also for Staphylococcus aureus and Klebsiella pneumoniae, where monocrobially infected patients of each type of infection also exhibited bacteriostatic activity (data not shown).

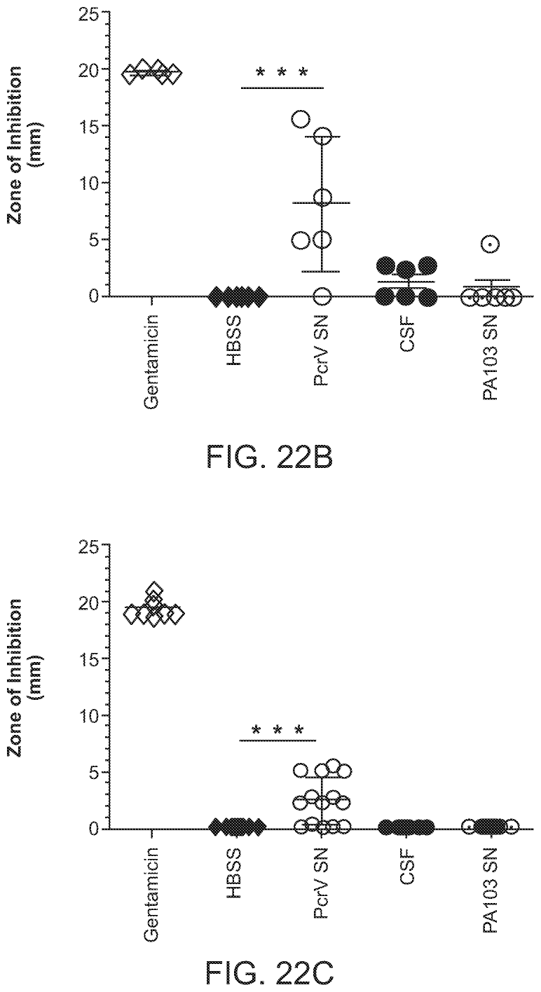

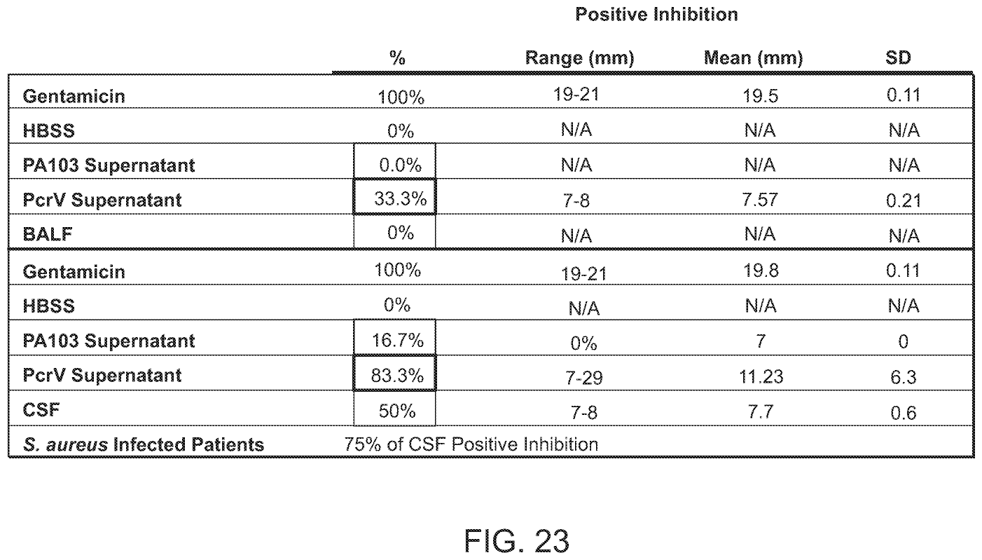

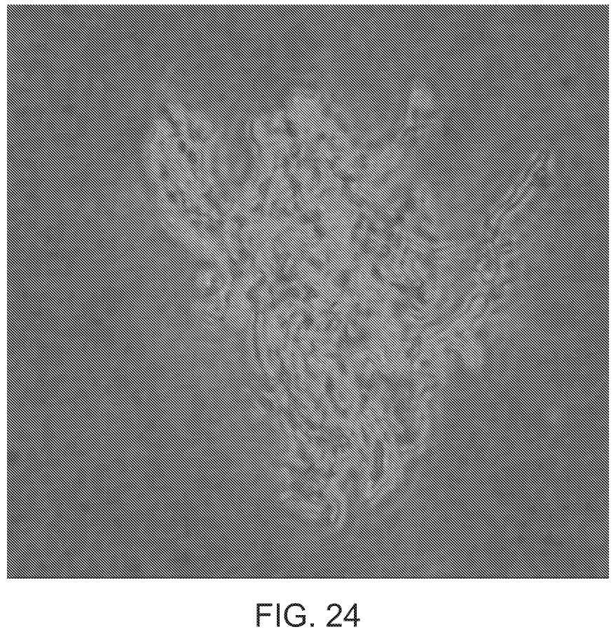

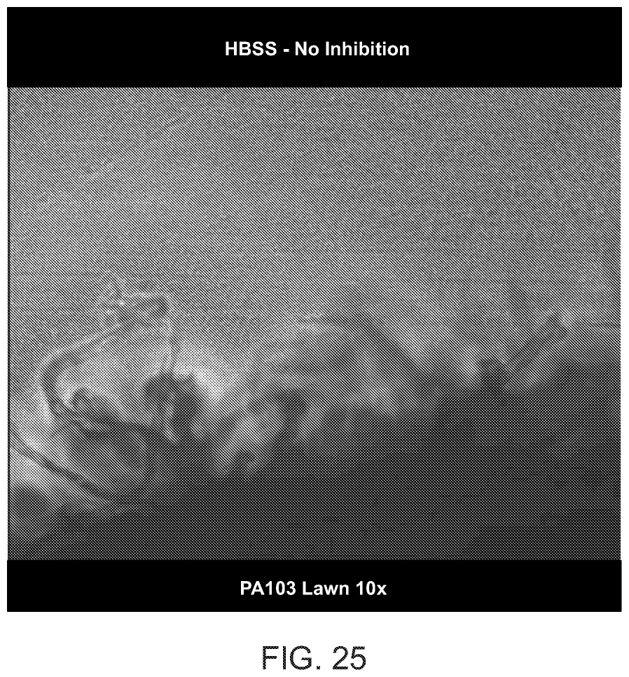

Next, the scope of clinically isolated infected cells capable of bacteriostatic amyloid production was examined. CSF-isolated cells showed some bacteriostatic activity (FIGS. 22A and 22B), while BALF-isolated cells did not (FIGS. 22A and 22C). These results were also tabulated in FIG. 23.

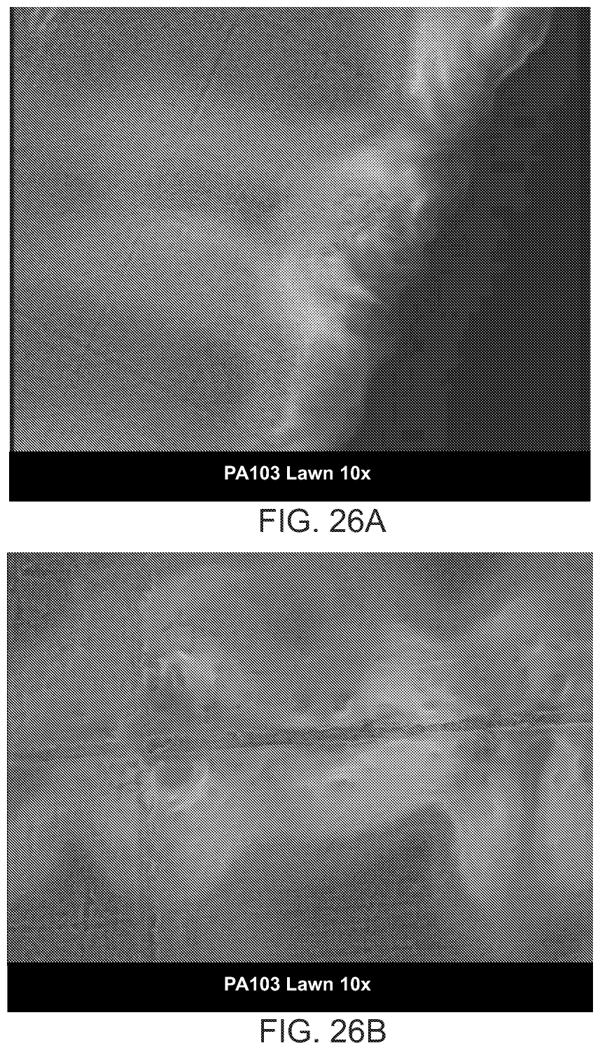

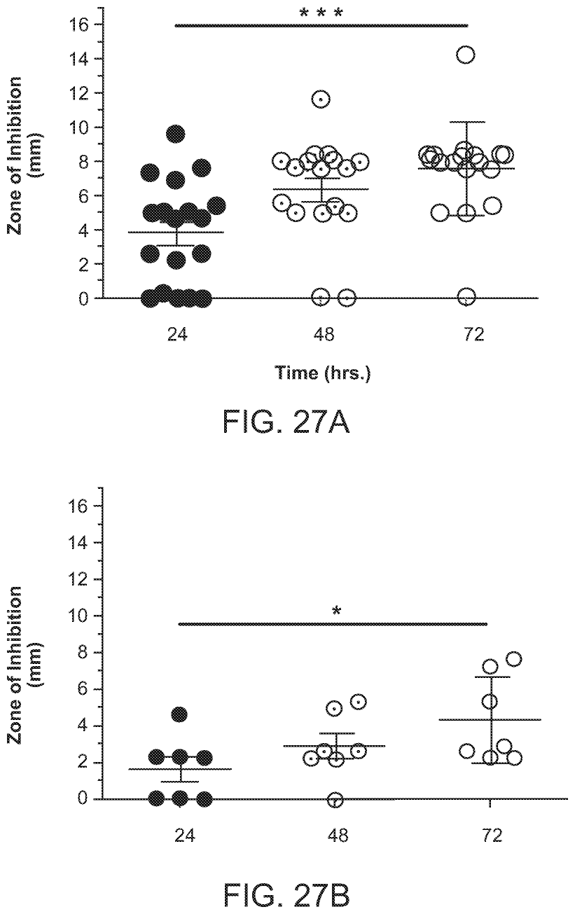

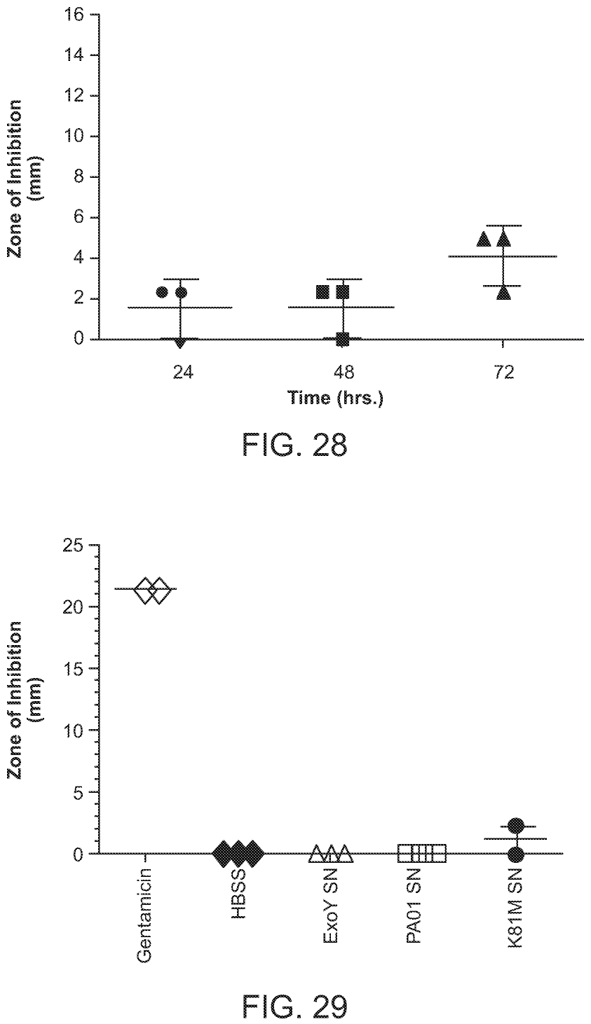

Advancing inhibition provoked by amyloid preparations was further observed for a ΔPcrV supernatant (PcrV SN)-treated PA103 lawn (FIGS. 26A and 26B). Time-dependence of the respective anti-microbial activities of ΔPcrV, PA103 (ExoU activity) and ExoY^{K81M} supernatants upon Pseudomonas spp. was further documented (FIGS. 27A, 27B and 28). Finally, the anti-microbial activity of ExoY supernatant upon Pseudomonas spp. was documented, as compared to gentamicin, HBSS (negative control), PA01 supernatant and ExoY^{K81M} supernatant (FIG. 29).

In sum, T3SS-induced amyloid proteins were herein identified as oligomers that (1) mediate cytotoxicity and (2) suppress antimicrobial activity. Meanwhile, the immunedepletion/neutralization of amyloid oligomers unexpectedly rescued the antimicrobial activity of endothelial amyloids post-infection. Pulmonary endothelial amyloids as described herein were therefore identified as inherently functional as antimicrobial agents.

### Example 3: Aβ Species and Oligomeric Amyloids Contribute to the Antimicrobial Amyloid Complex

Endothelial cells were infected with T3SS-deficient ΔPcrV to instigate antimicrobial amyloid release into the supernatant. Supernatants were collected, centrifuged, and filter-sterilized to render bacteria-free amyloid suspensions. Aliquots of ΔPcrV supernatant were then either neutralized with a single anti-amyloid antibody, or sequentially with more than one anti-amyloid antibody in a serial neutralization. Samples were standardized by protein to 50 μg/mL and applied to established bacterial lawns on YESCA Congo Red agar (Congo Red: 200 μg/mL, Coomassie Brilliant Blue R-250 200 μg/mL) to assess antimicrobial aggregation on a solid substrate. Images were taken at 72 hours and analyzed with a custom macro for ImageJ (NIH) to generate binary masks for quantification.

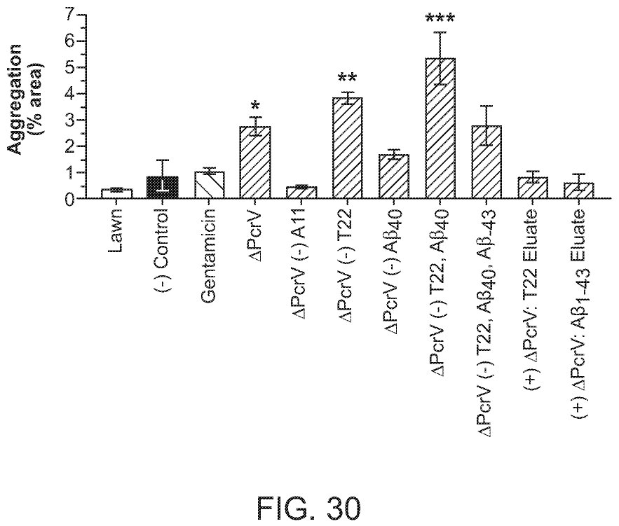

As shown in FIG. 30, A11 (pan-oligomeric amyloid) antibody neutralization ablated aggregation of bacteria [see ΔPcrV (−) A11] whereas T22 (anti-amyloid oligomer) antibody neutralization augmented the efficacy of the infection-derived antimicrobial amyloid complex [see ΔPcrV (−) T22].

Treatment with monoclonal anti-Aβ40 (11A50-B10) decreased aggregation [see ΔPcrV (−) Aβ₄₀] albeit when T22 and anti-Aβ antibodies were used sequentially, there was a significant improvement in the % area aggregated [see ΔPcrV (−) T22, Aβ₄₀]. Interestingly sequential neutralization of that sample with the anti-pan-Aβ antibody that recognizes all Aβ variants reduced the aggregation of bacteria [see see ΔPcrV (−) T22, Aβ₄₀, Aβ₁₋₄₃]. The elution and application of either T22 [(+) ΔPcrV: T22 Eluate] or pan-Aβ antibody-captured species [(+) ΔPcrV: Aβ₁₋₄₃ Eluate] failed to recapitulate the efficacy of the primary T3SS-infection generated antimicrobial amyloid complex. Taken together, these data suggest that oligomeric amyloids and Aβ species significantly contribute to the antimicrobial complex. However, most importantly, the majority of the effect cannot be attributed to Aβ alone, and the sequential neutralization of anti-oligomeric tau (T22-reactive species) and anti-Aβ₄₀ (11A50-B10-reactive species) [see ΔPcrV (−) T22, Aβ₄₀] was sufficient to significantly increase the antimicrobial capacity of the primary antimicrobial complex. n >3; 3-9 technical replicates for each independent experiment; mean±SEM; one-way ANOVA with Dunnett's post-hoc. *p<0.01, **p<0.001, ***p<0.0001.

### Example 4: Antimicrobial Amyloid Complex Prevents Biofilm Formation

T3SS-deficient mutant ΔPcrV was used to infect naïve endothelial cells as previously described to generate antimicrobial amyloid-rich supernatants. Supernatants were harvested, spun down, and filter-sterilized to remove bacteria and cell debris. Primary supernatants were then passaged to naïve endothelial monolayers and incubated for 4 hours. The primary supernatant was then removed, the monolayer rinsed 5× with HBSS, and clear DMEM applied prior to incubation for 20 hours to produce a secondary (2°) supernatant. Secondary supernatants were collected, centrifuged, and filter-sterilized prior to passaging to another naïve monolayer, and the process repeated, to generate a tertiary (3°) supernatant. To produce the heat-killed ExoY⁺, the virulent ExoY⁺ mutant was subjected to heating at 65° C. for 15 minutes to inactivate the bacteria. Heat-killed bacteria were then used to ‘infect’ monolayers for 7 hours. All supernatants were standardized to 50 μg/mL prior to use in the biofilm microtiter plate assay.

Overnight liquid cultures of lab strain PA01 and clinical isolate PA-815 (obtained from the BALF of an ICU patient diagnosed with monocrobial nosocomial pneumonia) were either aliquoted directly into round-bottomed polyvinylchloride (PVC) microplate wells (PA01 and PA-815, respectively), or incubated with standardized supernatant samples 1:1 for 15 minutes at room temperature. Round-bottom PVC plates were used to closely simulate endotracheal tube and in-dwelling catheter design and substrate. All samples were then diluted 1:100 with YESCA broth in the wells of the PVC plate and incubated for 24 hours. Following incubation, standard microtiter biofilm crystal violet assays were conducted (O'Toole. 2011. J. Vis. Exp. (47): 2437) with the exception of a methanol-fixation step following the first rinse, and 2 additional rinses following methanol-fixation. Absorbance was measured at 570 nm.

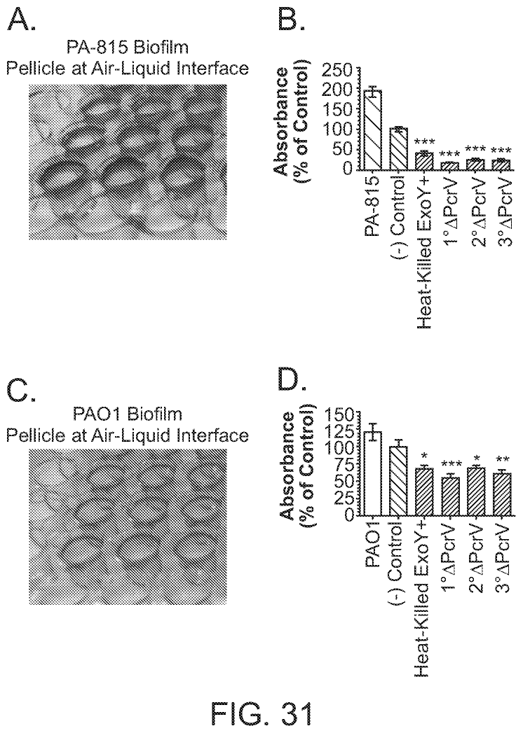

As shown in FIGS. 31A-D, endothelial amyloids markedly abrogate biofilm formation. Standard microtiter plate crystal violet biofilm assays using round-bottomed PVC plates to replicate endotracheal tube and in-dwelling catheter material and design were used. PVC plate images show a pellicle, or biofilm formed at the air-liquid interface by motile bacteria, stained purple around the upper portion of each well by nosocomial pneumonia isolate PA-815 (FIG. 31A) and lab strain PA01 (FIG. 31C). Both of these strains produce robust biofilms. FIGS. 31B and 31D demonstrate that T3SS-deficient ΔPcrV infection-derived supernatant significantly attenuated biofilm formation on PVC substrate (see 1° ΔPcrV FIGS. 31B and 31D, respectively), proving particularly effective in the prophylaxis of clinical isolate PA-815 generated biofilm as compared to the negative control (see see 1° ΔPcrV FIG. 31B). Importantly, subsequent generations of passaged amyloids (2° ΔPcrV and 3° PcrV FIGS. 31B and 31D, respectively) exhibit equivalent potency in the prevention of biofilm formation. n=10; 5 technical replicates for each independent experiment; mean±SEM; one-way ANOVA with Dunnett's post-hoc. *p<0.01, **p<0.001, ***p<0.0001.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for producing and harvesting an antimicrobial amyloid protein composition, the method comprising:
(a) contacting a mammalian cell in cell culture media with an infectious agent that:
does not possess a Type 3 Secretion System (T3SS) and/or does not possess T3SS-related exoenzymes or
is not capable of injecting T3SS-mediated exoenzymes into the cytosol of the mammalian cell,
thereby producing a first cell culture admixture;
(b) incubating the first cell culture admixture for an amount of time sufficient to induce the mammalian cell production and release of an antimicrobial amyloid protein complex into the cell culture media; and
(c) harvesting the cell culture media that harbors the antimicrobial amyloid protein complex,
thereby producing and harvesting an antimicrobial amyloid protein composition.

2. The method of claim 1, wherein the mammalian cell is selected from the group consisting of an endothelial cell and an epithelial cell.

3. The method of claim 1, wherein the mammalian cell is a Pulmonary Microvascular Endothelial Cell (PMVEC).

4. The method of claim 1, wherein the infectious agent is a bacteria, optionally a bacteria selected from the group consisting of a Gram-positive bacteria and a Gram-negative bacteria that does not possess a T3SS and/or T3SS-related exoenzymes or is not capable of injecting T3SS-mediated exoenzymes into the cytosol of the mammalian cell, optionally a Gram-negative bacteria that does not possess a T3SS and/or T3SS-related exoenzymes or is not capable of injecting T3SS-mediated exoenzymes into the cytosol of the mammalian cell selected from the group consisting of a Pseudomonas spp. bacteria and a Klebsiella pneumoniae bacterium, optionally a Gram-negative bacteria possessing a T3SS having reduced T3SS activity and/or reduced T3SS-related exoenzyme activity.

5. The method of claim 4, wherein the bacteria possessing a T3SS having reduced T3SS activity and/or reduced T3SS-related exoenzyme activity is a Pseudomonas aeruginosa bacteria.

6. The method of claim 4, wherein the bacteria possessing a T3SS having reduced T3SS activity and/or reduced T3SS-related exoenzyme activity is selected from the group consisting of a Pseudomonas aeruginosa ΔPcrV mutant and a Pseudomonas aeruginosa mutant possessing an ExoY cyclic nucleotidyl cyclase deficiency, optionally wherein the Pseudomonas aeruginosa mutant possessing an ExoY cyclic nucleotidyl cyclase deficiency is a Pseudomonas aeruginosa possessing an ExoY^{K81M} mutant.

7. The method of claim 1, wherein the cell culture comprising the antimicrobial amyloid protein complex is non-cytotoxic and is capable of biofilm degradation or inhibition or substantial attenuation of biofilm formation.

8. The method of claim 1, wherein step (b) comprises incubating the first cell culture admixture for an initial period of time and refreshing the cell culture media after the initial period of time, optionally wherein the initial period of time is about four hours, or is about five hours.

9. The method of claim 1, further comprising step (d) filter-sterilizing the cell culture comprising the antimicrobial amyloid protein complex.

10. The method of claim 10, further comprising steps (e)-(g):
(e) contacting a naïve mammalian cell with the harvested cell culture media comprising the antimicrobial amyloid protein complex of step (c) and additional cell culture media, thereby producing a second cell culture admixture;
(f) incubating the second cell culture admixture for an amount of time sufficient to induce in the naïve mammalian cell production and release of an antimicrobial amyloid protein complex into the cell culture media of the second cell culture admixture; and
(g) harvesting the cell culture media of the second cell culture admixture comprising the antimicrobial amyloid protein complex.

11. The method of claim 10, further comprising repeating steps (e) through (g) two or more times, optionally between two and five times.

12. The method of claim 1, wherein the incubating step further comprises depleting or neutralizing tau amyloid species and depleting or neutralizing Aβ amyloid species, optionally wherein the depleting or neutralizing comprises sequentially depleting or neutralizing Aβ amyloid species and then depleting or neutralizing tau amyloid species, or sequentially depleting or neutralizing tau amyloid species and then depleting or neutralizing Aβ amyloid species.

13. The method of claim 10, wherein the incubating steps (b) and/or (f) further comprise depleting or neutralizing tau amyloid species and depleting or neutralizing Aβ amyloid species, optionally wherein the depleting or neutralizing comprises depleting or neutralizing tau amyloid species and depleting or neutralizing Aβ amyloid species, optionally wherein the depleting or neutralizing comprises sequentially depleting or neutralizing Aβ amyloid species and then depleting or neutralizing tau amyloid species, or sequentially depleting or neutralizing tau amyloid species and then depleting or neutralizing Aβ amyloid species.

14. The method of claim 11, wherein the incubating steps (b) and/or (f) further comprise depleting or neutralizing tau amyloid species and depleting or neutralizing Aβ amyloid species, optionally wherein the depleting or neutralizing comprises sequentially depleting or neutralizing Aβ amyloid species and then depleting or neutralizing tau amyloid species, or sequentially depleting or neutralizing tau amyloid species and then depleting or neutralizing Aβ amyloid species.

15. An antimicrobial amyloid protein composition produced by the method of claim 1.

16. A pharmaceutical composition comprising an antimicrobial amyloid protein composition produced by the method of claim 1 and a pharmaceutically acceptable carrier.

17. A method for treating a subject having or at risk of developing a microbial infection comprising administering a pharmaceutical composition of claim 16 to the subject, thereby treating a subject having or at risk of developing a microbial infection.

18. The method of claim 17, wherein the microbial infection is a nosocomial infection, optionally wherein the nosocomial infection is a nosocomial staphylococcus infection, optionally a nosocomial pneumonia, optionally wherein the microbial infection is antibiotic resistant and/or multi-drug resistant (MDR).

19. A method for treating or preventing a nosocomial infection, a systemic infection, a superficial infection and/or a burn in a subject in need thereof, the method comprising administering to the subject a pharmaceutical composition of claim 16 thereby treating or preventing a nosocomial infection, a systemic infection, a superficial infection and/or a burn in the subject, optionally wherein:
the systemic infection is selected from the group consisting of sepsis and meningitis or
the superficial infection is a superficial infection of the central nervous system (CNS).

20. A composition for producing an antimicrobial amyloid protein complex comprising a mammalian cell infected with an infectious agent that:
does not possess a T3SS and/or does not possess T3SS-related exoenzymes or
is not capable of injecting T3SS-mediated exoenzymes into the cytosol of the mammalian cell.

21. The composition of claim 20, wherein the mammalian cell is selected from the group consisting of an endothelial cell and an epithelial cell.

22. The composition of claim 20, wherein the mammalian cell is a Pulmonary Microvascular Endothelial Cell (PMVEC) or Pulmonary Arterial Endothelial Cell (PAEC).

23. The composition of claim 20, wherein the infectious agent is a bacteria, optionally a bacteria selected from the group consisting of a Gram-positive bacteria and a Gram-negative bacteria that does not possess a T3SS and/or T3SS-related exoenzymes or is not capable of injecting T3SS-mediated exoenzymes into the cytosol of the mammalian cell, optionally a Gram-negative bacteria that does not possess a T3SS and/or T3SS-related exoenzymes or is not capable of injecting T3SS-mediated exoenzymes into the cytosol of the mammalian cell selected from the group consisting of a Pseudomonas spp. bacteria and a Klebsiella pneumoniae bacterium, optionally a Gram-negative bacteria possessing a T3SS having reduced T3SS activity and/or reduced T3SS-related exoenzyme activity.

24. The composition of claim 23, wherein the bacteria possessing a T3SS having reduced T3 SS activity is a Pseudomonas aeruginosa bacteria.

25. The composition of claim 23, wherein the bacteria possessing a T3SS having reduced T3SS activity and/or reduced T3SS-related exoenzyme activity is selected from the group consisting of a Pseudomonas aeruginosa ΔPcrV mutant and a Pseudomonas aeruginosa mutant possessing an ExoY cyclic nucleotidyl cyclase deficiency, optionally wherein the Pseudomonas aeruginosa mutant possessing an ExoY cyclic nucleotidyl cyclase deficiency is a Pseudomonas aeruginosa possessing an ExoY^{K81M} mutant.

26. The composition of claim 20, wherein the antimicrobial amyloid protein complex is non-cytotoxic and is capable of biofilm degradation or inhibition or substantial attenuation of biofilm formation.

27. A method for treating a microbial infection in a subject, the method comprising administering to the subject in need thereof an anti-amyloid antibody in an amount sufficient to deplete amyloid levels in the subject, thereby treating the microbial infection in a subject.

28. The method of claim 27, wherein the microbial infection is selected from the group consisting of a Pseudomonas aeruginosa bacteria, a Staphylococcus aureus bacteria and a Klebsiella pneumoniae bacterium.

29. The method of claim 28, wherein the Pseudomonas aeruginosa bacteria possesses an intact T3SS.

30. The method of claim 27, wherein the anti-amyloid antibody is capable of neutralizing or immunodepleting an amyloid oligomer in the subject.

31. The method of claim 27, wherein the anti-amyloid antibody is an anti-tau antibody, optionally an anti-tau oligomer antibody, optionally a monoclonal anti-tau antibody, optionally a monoclonal anti-tau antibody that binds the oligomeric conformation of tau, optionally a monoclonal anti-tau antibody selected from the group consisting of a T22 antibody, a TNT1 antibody and a TOC1 antibody.

32. The method of claim 27, wherein the anti-amyloid antibody is selected from the group consisting of an anti-amyloid Aβ antibody and a pan-anti-amyloid antibody and an anti-amyloid A antibody, optionally wherein the anti-amyloid antibody is a conformationally specific pan-amyloid antibody which optionally is OC, or an anti-amyloid antibody which optionally is pan-amyloid antibody A11, or an anti-Aβ antibody specific for the oligomeric conformation of Aβ, optionally wherein the anti-Aβ antibody specific for the oligomeric conformation of Aβ is a monoclonal or a polyclonal anti-Aβ antibody specific for unaggregated species and conformations of the oligomeric conformation of Aβ, optionally wherein the monoclonal anti-Aβ antibody specific for unaggregated species and conformations of Aβ is MOAB-2, or wherein the polyclonal anti-Aβ antibody targets any Aβ variant for Aβ is an Aβ 1-43 antibody.

33. The method of claim 27, wherein the anti-amyloid antibody is administered in combination with an antimicrobial peptide treatment.

34. A method for producing and collecting an antimicrobial amyloid protein composition comprising:
(a) culturing a mammalian cell in a medium;
(b) adding to the product of step (a) an infectious agent that:
does not possess a T3SS and/or does not possess T3SS-related exoenzymes or is not capable of injecting T3SS-mediated exoenzymes into the cytosol of the mammalian cell
in an amount sufficient to induce the mammalian cell to produce and release amyloid protein complexes into the medium, thereby producing a medium product comprising amyloid protein complexes;
(c) removing the medium product of step (b), thereby producing a mammalian cell product;
(d) culturing the mammalian cell product of step (c) in a fresh medium for a time length sufficient to allow for mammalian cell production and release of amyloid protein complexes into the medium, thereby producing a second medium product comprising amyloid protein complexes; and
(e) collecting the second medium product of (d),
thereby producing and collecting an antimicrobial amyloid protein composition.

35. The method of claim 34, wherein steps (b) and/or (d) further comprise depleting or neutralizing tau amyloid species and depleting or neutralizing Aβ amyloid species, optionally wherein the depleting or neutralizing comprises sequentially depleting or neutralizing Aβ amyloid species and then depleting or neutralizing tau amyloid species, or sequentially depleting or neutralizing tau amyloid species and then depleting or neutralizing Aβ amyloid species.

36. The method of claim 34, further comprising step (f) recovering the amyloid protein complexes from the second medium product of step (e).

37. A composition comprising an antimicrobial amyloid protein composition produced by the method of claim 34.

38. A composition comprising an antimicrobial amyloid protein composition produced by the method of claim 37.

39. A method for inhibiting or substantially attenuating formation of a biofilm on an in-dwelling catheter and/or endotracheal tube or a wound, degrading a biofilm present on an indwelling catheter and/or endotracheal tube or a wound, the method comprising applying the antimicrobial amyloid protein composition of claim 15.
